SEQUENCE LISTING (20 sequences)

SEQ ID NO: 1 - DNA, 20 bp, synthetic construct
  misc_feature 1..20: note = SERPINE1 (PAI-1) forward primer
  source 1..20: mol_type = other DNA
gccagtggaagactcccttc

SEQ ID NO: 2 - DNA, 20 bp, synthetic construct
  misc_feature 1..20: note = SERPINE1 (PAI-1) reverse primer
  source 1..20: mol_type = other DNA
gtgctgccgtctgatttgtg

SEQ ID NO: 3 - DNA, 20 bp, synthetic construct
  misc_feature 1..20: note = ANG forward primer
  source 1..20: mol_type = other DNA
tgggcgttttgttgttggtc

SEQ ID NO: 4 - DNA, 20 bp, synthetic construct
  misc_feature 1..20: note = ANG reverse primer
  source 1..20: mol_type = other DNA
ggcatcatagtgctgggtca

SEQ ID NO: 5 - DNA, 18 bp, synthetic construct
  misc_feature 1..18: note = SDC1 forward primer
  source 1..18: mol_type = other DNA
gctctggctctggctgtg

SEQ ID NO: 6 - DNA, 20 bp, synthetic construct
  misc_feature 1..20: note = SDC1 reverse primer
  source 1..20: mol_type = other DNA
atcttgcaaagcacctgcac

SEQ ID NO: 7 - DNA, 20 bp, synthetic construct
  misc_feature 1..20: note = SERPINA1 (A1AT) forward primer
  source 1..20: mol_type = other DNA
gtaccctcaaccagccagac

SEQ ID NO: 8 - DNA, 20 bp, synthetic construct
  misc_feature 1..20: note = SERPINA1 (A1AT) reverse primer
  source 1..20: mol_type = other DNA
tgtccccgaagttgacagtg

SEQ ID NO: 9 - DNA, 20 bp, synthetic construct
  misc_feature 1..20: note = MMP9 forward primer
  source 1..20: mol_type = other DNA
cagtccacccttgtgctctt

SEQ ID NO: 10 - DNA, 20 bp, synthetic construct
  misc_feature 1..20: note = MMP9 reverse primer
  source 1..20: mol_type = other DNA
cgactctccacgcatctctg

SEQ ID NO: 11 - DNA, 20 bp, synthetic construct
  misc_feature 1..20: note = MMP10 forward primer
  source 1..20: mol_type = other DNA
cctgggttttcctccaacca

SEQ ID NO: 12 - DNA, 20 bp, synthetic construct
  misc_feature 1..20: note = MMP10 reverse primer
  source 1..20: mol_type = other DNA
gtctagggaagccttgctcc

SEQ ID NO: 13 - DNA, 20 bp, synthetic construct
  misc_feature 1..20: note = CA9 (CAIX) forward primer
  source 1..20: mol_type = other DNA
gcctttgaatgggcgagtga

SEQ ID NO: 14 - DNA, 20 bp, synthetic construct
  misc_feature 1..20: note = CA9 (CAIX) reverse primer
  source 1..20: mol_type = other DNA
gccaaaaaccagggctagga

SEQ ID NO: 15 - DNA, 20 bp, synthetic construct
  misc_feature 1..20: note = APOE forward primer
  source 1..20: mol_type = other DNA
cgcttttgggattacctgcg

SEQ ID NO: 16 - DNA, 20 bp, synthetic construct
  misc_feature 1..20: note = APOE reverse primer
  source 1..20: mol_type = other DNA
tcatggtctcgtccatcagc

SEQ ID NO: 17 - DNA, 20 bp, synthetic construct
  misc_feature 1..20: note = VEGFA forward primer
  source 1..20: mol_type = other DNA
gtcctggagcgttccctgtg

SEQ ID NO: 18 - DNA, 20 bp, synthetic construct
  misc_feature 1..20: note = VEGFA reverse primer
  source 1..20: mol_type = other DNA
ccttgcaacgcgagtctgtg

SEQ ID NO: 19 - DNA, 20 bp, synthetic construct
  misc_feature 1..20: note = CXCL8 (IL8) forward primer
  source 1..20: mol_type = other DNA
cactgcgccaacacagaaat

SEQ ID NO: 20 - DNA, 22 bp, synthetic construct
  misc_feature 1..22: note = CXCL8 (IL8) reverse primer
  source 1..22: mol_type = other DNA
atgaattctcagccctcttcaa

(19) United States
(12) Patent Application Publication
(10) Pub. No.: US 2025/0305057 A1
(43) Pub. Date: Oct. 2, 2025

(54) BLADDER CANCER BIOMARKERS AND METHODS OF USE

(71) Applicant: NONAGEN BIOSCIENCE CORPORATION, Los Angeles, CA (US)

(72) Inventors: Charles J. ROSSER, Stevenson Ranch, CA (US); Steve GOODISON, Jacksonville, FL (US)

(21) Appl. No.: 18/869,727

(22) PCT Filed: May 26, 2023
(86) PCT No.: PCT/US2023/067562

§ 371 Date: Nov. 26, 2024

Related U.S. Application Data
(60) Provisional application No. 63/346,468, filed on May 27, 2022.
(60) Provisional application No. 63/483,679, filed on Feb. 7, 2023.

(51) Int. Cl. C12Q 1/6886; G01N 33/574; G16B 25/10; G16B 40/00; G16H 50/20

(52) U.S. Cl. CPC C12Q 1/6886; G01N 33/57407; G16B 25/10; G16B 40/00; G16H 50/20; C12Q 2600/118; C12Q 2600/158; G01N 2333/475; G01N 2333/515; G01N 2333/5421; G01N 2333/70596; G01N 2333/775; G01N 2333/8125; G01N 2333/8132; G01N 2333/96494; G01N 2333/988

(57) ABSTRACT

Compositions, kits, and methods for the prognosis of bladder cancer in a subject are provided by detecting in tumor tissue a combination of biomarkers consisting of ANG, A1AT, APOE, CA9, IL8, MMP9, MMP10, PAI-1, SDC1 and VEFGA.

### CROSS REFERENCE TO RELATED APPLICATIONS

This instant application claims priority to U.S. Provisional Application No. 63/346,468, filed on May 27, 2022, and U.S. Provisional Patent Application No. 63/483,679, filed Feb. 7, 2023, the contents of each which are hereby incorporated by reference in their entireties.

### REFERENCE TO SEQUENCE LISTING SUBMITTED AS A COMPLIANT XML 1.0 FORMAT FILE (.xml)

Pursuant to the EFS-Web legal framework and 37 CFR §§ 1.821-825 (see MPEP § 2442.03(a)), Rule 30 EPC, and § 11 PatV, an electronic sequence listing compliant with WIPO standard ST.26 in the form of an XML 1.0 format file (entitled “300047-005977_Sequence_Listing.xml” created on May 26, 2023, and 26,185 bytes in size) is submitted concurrently with the instant application, and the entire contents of the sequence listing are incorporated herein by reference. For the avoidance of doubt, if discrepancies exist between the sequences mentioned in the specification and the electronic sequence listing, the sequences in the specification shall be deemed to be the correct ones.

### BACKGROUND

### 1. Field

The present invention is directed to compositions, kits, and methods of cancer detection, and, in particular, to such compositions, kits, and methods in the prognosis of bladder cancer. In addition, such compositions, kits, and methods are useful as an adjunct to pathological assessments.

### 2. Description of Related Art

Bladder cancer is among the five most common malignancies worldwide. An estimated 83,730 newly diagnosed cases of bladder cancer and 17,200 deaths from bladder cancer will occur in 2021 in the US alone. Siegel et al. (2021) CA Cancer J Clin 71(1): 7-33. Both the absolute numbers of cases and deaths from bladder cancer have increased by 57 and 41%, respectively, since 2000. Siegel et al. (2021) CA Cancer J Clin 71(1): 7-33; Greenlee et al. (2000) CA Cancer J Clin 50(1): 7-33. When detected early (i.e., NMIBC or stage 1), the 5-year survival rate is approximately 94%, compared to at best 50% 5-year survival rate when the disease is noted to be MIBC (stage 2) and less than 20% 5-year survival rate when the disease is metastatic (stages 3 and 4). Brausi et al. J Urol. (2011) 186(6): 2158-67; Stenzl et al. Eur Urol. (2011) 59(6): 1009-18; Calabro et al. Curr Opin Support Palliat Care. (2012) 6(3): 304-9; Sternberg et al. J Clin Oncol. (2001) 19(10): 2638-46.

Oncologists have several treatment options available to them, including surgery, radiation, chemotherapeutic drugs and immune-oncology agents. The best likelihood of good treatment outcome requires that patients be assigned to optimal available cancer treatment, and that this assignment be made as quickly as possible following diagnosis.

There exists a need in the art for early, rapid detection of cancer to improve clinical outcomes.

### BRIEF SUMMARY

In an embodiment, a method for predicting the likelihood of long-term survival of a bladder cancer patient can comprise (a) obtaining a biological sample from a patient; (b) isolating mRNA from the biological sample; (c) determining the level of the mRNA of ANG, A1AT, APOE, CA9, IL8, MMP9, MMP10, PAI-1, SDC1 and VEFGA in the biological sample; (d) normalizing the mRNA level against a level of at least one reference mRNA transcript in the sample to provide a normalized ANG, A1AT, APOE, CA9, IL8, MMP9, MMP10, PAI-1, SDC1 and VEFGA mRNA level; (e) comparing the normalized ANG, A1AT, APOE, CA9, IL8, MMP9, MMP10, PAI-1, SDC1 and VEFGA mRNA level to a normalized ANG, A1AT, APOE, CA9, IL8, MMP9, MMP10, PAI-1, SDC1 and VEFGA mRNA level in reference bladder tumor samples; and (f) predicting the likelihood of long-term survival without the recurrence of bladder cancer, wherein increased ANG, A1AT, APOE, CA9, IL8, MMP9, MMP10, PAI-1, SDC1 and VEFGA mRNA levels is indicative of a reduced likelihood of long-term survival without recurrence of bladder cancer. The biomarkers can consist of ANG, A1AT, APOE, CA9, IL8, MMP9, MMP10, PAI-1, SDC1, and VEFGA.

In an embodiment, a method for detecting bladder cancer biomarkers can comprise: (a) obtaining a biological sample from a patient; (b) isolating RNA from the biological sample; and (c) determining the level of ANG, A1AT, APOE, CA9, IL8, MMP9, MMP10, PAI-1, SDC1 and VEFGA mRNA in the biological sample.

In an embodiment, a method of classifying test data, the test data comprising RNA expression data, the method can comprise: (a) accessing, using at least one processor, an electronically stored set of training data vectors, each training data vector representing an individual cancer patient and comprising a RNA expression data for the respective cancer patient, each training data vector further comprising a classification with respect to the expression level of a biomarker selected from the group consisting of ANG, A1AT, APOE, CA9, IL8, MMP9, MMP10, PAI-1, SDC1, VEFGA, and combinations thereof, (b) training an electronic representation of a classification system, using the electronically stored set of training data vectors; (c) receiving, at the at least one processor, test data comprising RNA expression data; (d) evaluating, using the at least one processor, the test data using the electronic representation of the classification system; and (e) outputting a classification of the test data concerning the likelihood of long-term survival without the recurrence of bladder cancer based on the evaluating step. The biomarkers can consists of ANG, A1AT, APOE, CA9, IL8, MMP9, MMP10, PAI-1, SDC1, and VEFGA.

In an embodiment, the classification system can be AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, Naïve Bayes, neural nets, penalized logistic regression, Random Forests, ridge regression, support vector machines, or an ensemble thereof. The classification system can be an ensemble of classification systems.

In an embodiment, the mRNA level can be determined by microarray analysis, RNAseq, RT-PCR, RT-qPCR, quantitative PCR (qPCR), Northern blot analysis, dot blotting, Southern blot analysis, RNA sequencing, fluorescence in situ hybridization (FISH), or a combination thereof. The mRNA can be determined by quantitative PCR (qPCR). The microarray can comprise cDNA of biomarker selected from the group consisting of ANG, A1AT, APOE, CA9, IL8, MMP9, MMP10, PAI-1, SDC1, VEFGA, and combinations thereof. The microarray can comprise cDNA can be fixed to a substrate. The biomarkers can consists of ANG, A1AT, APOE, CA9, IL8, MMP9, MMP10, PAI-1, SDC1, and VEFGA.

In an embodiment, the determination step can use a primer selected from the group consisting of SEQ ID NO: 1, 2, 3, 4, 5, 6, 7, 8, 9, 10, 11, 12, 13, 14, 15, 16, 17, 18, 19, 20, or combinations thereof. The determination step can use a primer pair selected from the group consisting of SEQ ID NO: 1 and 2; 3 and 4; 5 and 6; 7 and 8; 9 and 10; 11 and 12; 13 and 14; 15 and 16; 17 and 18; 19 and 20; or a combination thereof.

In an embodiment, the determination step can use a label nucleic acid probe. The label can be a radioactive label, a fluorescent label, an enzyme, a chemiluminescent tag, a colorimetric tag, or a combination thereof.

In an embodiment, the RNA can be sequenced.

In an embodiment, the biological sample can be blood, serum, whole, blood, circulating tumor cells, tumor cells, plasma, urine, tissue, tumor, or a combination thereof. The biological sample can be tissue, optionally tumor tissue. The tissue can be a fixed, wax-embedded tissue sample.

In an embodiment, the level of the amplicon of the RNA transcript of ANG, A1AT, APOE, CA9, IL8, MMP9, MMP10, PAI-1, SDC1 and VEFGA can be represented as a threshold cycle (Ct) value and the normalized ANG, A1AT, APOE, CA9, IL8, MMP9, MMP10, PAI-1, SDC1 and VEFGA amplicon level is represented as a normalized Ct value.

In an embodiment, the reference bladder cancer samples can comprise at least 30 bladder cancer samples.

In an embodiment, the method can further comprise detecting and quantifying at least one additional biomarker of a urogenital-related cancer type in the biological sample or in a different biological sample.

In an embodiment, the method can further comprise detecting and quantifying at least one additional biomarker of a different cancer type in the biological sample or in a different biological sample.

In an embodiment, the method can be performed at several time points or intervals as part of monitoring of the subject at least one of before, during, and after treatment of the cancer.

In an embodiment, the method can further comprise the step of preparing a report indicating that the patient has an increased or decreased likelihood of long-term survival without bladder cancer.

In an embodiment, a non-transitory computer readable medium storing an executable program can comprise instructions to perform the methods described herein.

In an embodiment, a system, comprising: a server comprising at least one processor and memory can comprise computer-readable instructions which when executed by the processor cause the processor to perform the steps comprising: receiving mRNA expression data from a computer terminal that is located remotely from the server; processing the mRNA expression data using a classification system.

In an embodiment, a method for detecting upper tract urothelial carcinoma (UTUC) biomarker can comprise (a) obtaining a biological sample from a subject; (b) contacting a biological sample obtained from a subject with a panel of binding agents, wherein said panel comprises binding agents that bind to, and form a complex, with proteins selected from the group consisting of ANG, A1AT, APOE, CA9, IL8, MMP9, MMP10, PAI-1, SDC1, VEFGA, and combinations thereof; and (c) detecting the presence and quantity of the protein-binding agent complexes that form in the biological sample. The biomarkers can consists of ANG, A1AT, APOE, CA9, IL8, MMP9, MMP10, PAI-1, SDC1, and VEFGA.

In an embodiment, a method of classifying test data, the test data comprising protein expression data, the method can comprise: (a) accessing, using at least one processor, an electronically stored set of training data vectors, each training data vector representing an individual cancer patient and comprising a protein expression data for the respective cancer patient, each training data vector further comprising a classification with respect to the expression level of a biomarker selected from the group consisting of ANG, A1AT, APOE, CA9, IL8, MMP9, MMP10, PAI-1, SDC1, VEFGA, and combinations thereof, (b) training an electronic representation of a classification system, using the electronically stored set of training data vectors; (c) receiving, at the at least one processor, test data comprising protein expression data; (d) evaluating, using the at least one processor, the test data using the electronic representation of the classification system; and (e) outputting a classification of the test data concerning the likelihood of upper tract urothelial carcinoma (UTUC) based on the evaluating step. The classification system can be AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, Naïve Bayes, neural nets, penalized logistic regression, Random Forests, ridge regression, support vector machines, or an ensemble thereof. The biomarkers can consist of ANG, A1AT, APOE, CA9, IL8, MMP9, MMP10, PAI-1, SDC1, and VEFGA. The classification system can be an ensemble of classification systems.

In an embodiment, a subject can be diagnosed with UTUC.

In an embodiment, a sample can be obtained from a subject who has at least one symptom of UTUC.

In an embodiment, the biological sample can be blood, serum, whole blood, circulating tumor cells, tumor cells, plasma, urine, tissue, tumor, or a combination thereof. The biological sample can be blood, urine, plasma, or a combination thereof. The biological sample can be urine.

In an embodiment, the binding agent can be an antibody or an antibody fragment. The binding agent can be an antibody. The binding agent can be a monoclonal antibody. The binding agent can be a polyclonal antibody.

In an embodiment, an array can comprise a biomarker selected from the group consisting of ANG, A1AT, APOE, CA9, IL8, MMP9, MMP10, PAI-1, SDC1, VEFGA, and combinations thereof fixed to a substrate. The biomarkers can consist of ANG, A1AT, APOE, CA9, IL8, MMP9, MMP10, PAI-1, SDC1, and VEFGA. The biomarker can be an mRNA transcript. The biomarker can be a cDNA of the mRNA transcript. The biomarker can be a peptide.

In an embodiment, a kit can comprise nucleic acid primers that specifically bind comprising a biomarker selected from the group consisting of ANG, A1AT, APOE, CA9, IL8, MMP9, MMP10, PAI-1, SDC1, VEFGA, and combinations. The biomarkers can consist of ANG, A1AT, APOE, CA9, IL8, MMP9, MMP10, PAI-1, SDC1, and VEFGA.

In an embodiment, a kit can comprise antibodies that specifically bind comprising a biomarker selected from the group consisting of ANG, A1AT, APOE, CA9, IL8, MMP9, MMP10, PAI-1, SDC1, VEFGA, and combinations. The biomarkers can consist of ANG, A1AT, APOE, CA9, IL8, MMP9, MMP10, PAI-1, SDC1, and VEFGA.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The advantages and features of the present invention will become better understood with reference to the following more detailed description taken in conjunction with the accompanying drawings in which:

FIG. 1 depicts an exemplary methodical approach utilized by the inventors to identify a diagnostic bladder cancer signature.

FIG. 2 depicts the single cell RNA sequencing of 25 human bladder cancers.

FIG. 3A-B depicts a (A) heatmap illustrating application of each of the 10 biomarkers associated with Oncuria™ in stratifying luminal vs. basal tumors within the TCGA cohort. Blue to Brown shows a trend from low to high gene expression. (B) Gene expression results of the individual biomarkers from the bladder cancer signature related to luminal vs. basal subtype.

FIG. 4 depicts the association of the individual 10 analytes with bladder cancer outcomes—TCGA.

FIG. 5 depicts the association of the combined 10 analytes with bladder cancer outcomes—TCGA

FIG. 6 depicts the association of the individual 10 analytes with bladder cancer outcomes—Black cohort.

FIG. 7 depicts the association of the combined 10 analytes with bladder cancer outcomes—Black cohort.

FIG. 8 depicts the association of the individual 10 analytes with bladder cancer outcomes—GSE 32894.

FIG. 9 depicts the association of the combined 10 analytes with bladder cancer outcomes—GSE32894.

FIG. 10 depicts the association of the individual 10 analytes with bladder cancer outcomes—GSE48075.

FIG. 11 depicts the association of the combined 10 analytes with bladder cancer outcomes—GSE48075.

FIG. 12 depicts the Kaplan-Meier survival curves for high vs. low expression of the biomarker signature in TCGA cohort; insert depicts TCGA analyzed by the consensus model.

FIG. 13A-C depicts Kaplan-Meier survival curves for high vs. low expression of the combined biomarker signature described herein in (A) GSE87304 cohort; insert depicts GSE87304 analyzed by the consensus subtyping system, (B) GSE48075 cohort; insert depicts GSE48075 analyzed by the MDA subtyping system and (C) GSE32894 cohort; insert depicts GSE32894 analyzed by the model reported in the associated GSE32894 manuscript (Damrauer et al. Proc Natl Acad Sci USA (2014) 111: 3110-3115; Choi et al. Cancer Cell (2014) 25: 152-165; Seiler et al. Eur Urol (2017) 72: 544-554).

FIG. 14 depicts a heatmap illustrating application of the 10 biomarkers of ONCURIA associated with the 6 consensus molecular subtype in the TCGA cohort. Blue to brown shows a trend from low to high gene expression.

FIG. 15 depicts comparison of urine concentrations of the 10 protein urinary biomarkers in UTUC and controls. Median levels are depicted by horizontal lines.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments and is not intended to be limiting. Instead, the scope of the present disclosure will be established by the appended claims.

Bladder Cancer Biomarkers with Prognostic Value

The present disclosure relates to a select set of genes, the expression of which has prognostic value, specifically with respect to disease-free survival, for example, in bladder cancer.

Diagnostic tests used in clinical practice are based on a single analyte, and therefore do not capture the potential value of knowing relationships between multiple biomarkers. Given the redundancy of signaling pathways, the cross-talk between molecular networks, and the oligoclonality of tumors, single biomarker assays lack adequate power to base critical diagnostic decisions. The inventors discovered a panel of RNA biomarkers that show unexpectedly improved prognosis of bladder cancer detection of tumor tissue.

Bladder cancer is a biologically heterogeneous disease with variable clinical presentations, outcomes, and responses to therapy. Thus, the clinical utility of single biomarkers for the detection and prediction of biological behavior of bladder cancer is limited. The inventors identified and validated a bladder cancer diagnostic signature comprised of 10 biomarkers ((ANG, APOE, A1AT, CA9, IL8, MMP9, MMP10, PAI1, SDC1 and VEGFA) and that may be incorporated into a multiplex immunoassay bladder cancer test. The inventors demonstrated that these 10 biomarkers can assist in the prediction of bladder cancer clinical outcomes. Tumor gene expression and patient survival data from bladder cancer cases from The Cancer Genome Atlas (TCGA) were analyzed. Alignment between the mRNA expression of 10 biomarkers and the TCGA 2017 subtype classification was assessed. Kaplan-Meier analysis of multiple gene expression datasets indicated that high expression of the combined 10 biomarkers correlated with a significant reduction in overall survival. The analysis of three independent, publicly available gene expression datasets confirmed that multiplex prognostic models outperformed single biomarkers. Eight of the 10 biomarkers (APOE, A1AT, IL8, MMP9, MMP10, PAI1, SDC1 and VEGFA) were significantly associated with either luminal or basal molecular subtypes and so the test has the potential to assist in the prediction of clinical outcome.

Bladder cancer is a biologically heterogeneous disease with variable clinical presentation, response to therapy and clinical outcome. The molecular complexity of bladder cancer has restricted the clinical utility of tests that rely on single features or biomarkers for the detection and prediction of bladder cancer behavior. The emergence of high-throughput molecular profiling technologies has enabled the development of multiplex molecular signatures with potential use for diagnosis, staging, prognostication and therapeutic decision making. There are currently two FDA-approved multiplex molecular tests for bladder cancer, UroVysion and the Immunocyt/Ucyt+Test, but their clinical utility has been impacted by limited sensitivity and specificity.

A multiplex immunoassay that quantitatively monitors a bladder cancer-associated diagnostic signature can comprise 10 protein biomarkers (ANG, APOE, A1AT, CA9, IL8, MMP9, MMP10, PAI1, SDC1 and VEGFA). In a series of studies, the molecular signature was developed and tested for the non-invasive detection of bladder cancer through urinalysis. In addition, immunostaining studies in excised bladder tumor tissues showed that expression of the these 10 biomarkers was increased in neoplastic over benign urothelium and high levels were associated with reduced overall patient survival.

The molecular subtyping of a range of solid tumors has emerged as a valuable tool for the classification of patients into genetically homogenous groups to guide clinical management. A number of subtyping schemes have been proposed for bladder cancer with varying levels of complexity. The inventors analyzed a series of gene expression datasets from TCGA and the Gene Expression Omnibus (GEO) to evaluate the potential utility of the 10 biomarkers (ANG, APOE, A1AT, CA9, IL8, MMP9, MMP10, PAI1, SDC1 and VEGFA) for the molecular subtyping of bladder cancer and the prediction of clinical outcome.

RNA-based tests have the disadvantages of RNA degradation and it is difficult to obtain fresh tissue samples from patients for analysis. Fixed paraffin-embedded tissue is more readily available and methods may be used to detect and extract higher quantity and quality of RNA from fixed tissue. The microarray can comprise cDNA of biomarker selected from the group consisting of ANG, A1AT, APOE, CA9, IL8, MMP9, MMP10, PAI-1, SDC1, VEFGA, and combinations thereof. The microarray can comprise cDNA can be fixed to a substrate.

The classification of bladder cancer by RNA gene expression analysis focuses on improving and refining a classification typically seen in bladder cancer, and have not provided any new insights into bladder cancer biology or the relationships of the differentially expressed genes and nor do the studies successfully link the findings to improving the clinical outcome of cancer therapy.

The challenge of cancer treatment remains to target specific treatment regimens to pathogenically distinct tumor types, and ultimately personalize tumor treatment in order to maximize outcome. The methods described herein provide tests that simultaneously provide prognostic information about patient clinical outcomes, for example, for bladder cancer, the biology of which is poorly understood.

The classification of the biomarkers selected by the inventors was trained on archived paraffin-embedded biopsy material to test all markers in the set, and therefore is compatible with the most widely available type of biopsy material. The methods described herein are also compatible with several different methods of tumor tissue harvest, for example, circulating tumor cells. Further, for each member of the gene set, the methods described herein specify oligonucleotide sequences that can be used in the test.

Cancer biomarkers (also called tumor biomarkers) are molecules such as DNA, RNA, metabolites, hormones, enzymes, and immunoglobulins found in the body that are associated with cancer and whose measurement or identification is useful in patient clinical management. They can be products of the cancer cells themselves, or of the body in response to cancer or other conditions. Most cancer biomarkers are RNA. As with other cancer biomarkers, the biomarkers described herein can be used for a variety of purposes, such as: screening a healthy population or a high-risk population for the presence of bladder cancer; making a diagnosis of bladder cancer or of a specific type of bladder cancer; determining the prognosis of a subject; and predicting/monitoring the course in a subject in remission or while receiving surgery, radiation, chemotherapy, or other cancer treatment.

The methods described herein may be used in the prognosis, prediction, and/or monitoring of cancer, optionally bladder cancer, can be performed at several time points or intervals, as part of monitoring of the subject before, during, or after treatment of the cancer.

A method for prognostic evaluation of a subject having, or suspected of having, cancer, optionally bladder cancer, can comprise: (a) determining the level of one or more cancer biomarkers listed in Table 1 in a biological sample obtained from the subject; (b) comparing the level determined in step (a) to a level or range of the one or more cancer biomarkers known to be present in a biological sample obtained from a normal subject that does not have cancer; and (c) determining the prognosis of the subject based on the comparison of step (b), wherein a high level of the one or more cancer biomarkers in step (a) indicates a more aggressive form of cancer and, therefore, a poor prognosis. The biomarker can comprise one or more nucleotides or polypeptides encoded by the nucleic acids listed in Table 1.

A method of predicting the likelihood of long-term survival of a bladder cancer patient, can comprise determining the expression level of one or more prognostic RNA transcripts or their expression products in a bladder cancer tissue sample obtained from the patient, normalized against the expression level of all RNA transcripts or their products in the bladder cancer tissue sample, or of a reference set of RNA transcripts or their expression products, wherein the prognostic RNA transcript is the transcript of one or more genes selected from the group consisting of: ANG, A1AT, APOE, CA9, IL8, MMP9, MMP10, PAI-1, SDC1 and VEFGA that collectively an increase indicates a decreased likelihood of long-term survival without bladder cancer recurrence.

In the methods described herein, the expression levels of at least two, or at least 5, or 10 of the prognostic RNA transcripts or their expression products can be determined.

In the methods described herein, the method can comprise the determination of the expression levels of all prognostic RNA transcripts or their expression products. A preferred subset of RNA transcripts can comprise ANG, A1AT, APOE, CA9, IL8, MMP9, MMP10, PAI-1, SDC1 and VEFGA that collectively an increase indicates a decreased likelihood of long-term survival without bladder cancer recurrence. The bladder cancer can be invasive bladder carcinoma. The RNA can be isolated from a fixed, wax-embedded bladder cancer tissue specimen of the patient. Isolation may be performed by any technique known in the art, for example from biopsy tissue or transurethral resection bladder tumor or fine needle aspirate cells or cystectomy tissue. The RNA can be isolated from circulating tumor cells of the patient. Isolation may be performed by any technique known in the art. See, e.g., Gjerde et al. “RNA Purification and Analysis: Sample Preparation, Extraction, Chromatography” (1^{st} Ed) (2009) Wiley-VCH.

A method of predicting the likelihood of long-term survival of a patient diagnosed with invasive bladder cancer can comprise:
(a) determining the expression levels of the RNA transcripts or the expression products of genes or a gene set selected from the group consisting of: ANG, A1AT, APOE, CA9, IL8, MMP9, MP10, PAI-1, SDC1 and VEFGA (Table 1);
(b) subjecting the data obtained in step (1) to statistical analysis; and
(c) determining whether the likelihood of said long-term survival has increased or decreased.

TABLE 1
List of 10 analytes comprising bladder cancer diagnostic signature

| | | | | UniProtKB/ |
|---|---|---|---|---|
| Gene | | NCBI | TaqMan | Swiss- |
| Symbol | Gene Name | Gene ID | Assay ID | Prot ID |
| CA9 | carbonic anhydrase IX | 768 | Hs00154208_m1 | Q16790 |
| MMP10 | matrix metallopeptidase 10 | 4319 | Hs00233987_m1 | P09238 |
| ANG | angiogenin, ribonuclease, RNase A | 283 | Hs01590076_m1 | P03950 |
| | family, 5 | | | |
| SERPINE1 | serpin peptidase inhibitor, clade E, | 5054 | Hs01126606_m1 | P05121 |
| | member 1 | | | |
| MMP9 | matrix metallopeptidase 9 | 4318 | Hs00234579_m1 | P14780 |
| IL8 | interleukin 8 | 3576 | Hs00174103_m1 | P10145 |
| VEGFA | vascular endothelial growth factor A | 7422 | Hs00900055_m1 | P15692 |
| APOE | apolipoprotein E | 348 | Hs00171168_m1 | P02649 |
| SERPINA1 | alpha-1-antitrypsin | 5265 | Hs00165475 | P01009 |
| SDC1 | syndecan 1 | 6382 | Hs04966523 | P18827 |

The gene sequences listed in Table 2 and a PCR primer-probe set listed in Table 3 may be used to detect and/or quantitate the biomarkers in the methods described herein.

TABLE 2
Sequence ID Gene Accession of 10 analytes comprising
bladder cancer diagnostic signature

| Gene | | | |
|---|---|---|---|
| Symbol | HGNC ID | RefSeq ID | Ensembl ID |
| CA9 | HGNC: 1383 | NM_001216 | ENSG00000107159 |
| MMP10 | HGNC: 7156 | NM_002425 | ENSG00000166670 |
| ANG | HGNC: 483 | NM_001097577 | ENSG00000214274 |
| SERPINE1 | HGNC: 8583 | NM_000602 | ENSG00000106366 |
| MMP9 | HGNC: 7176 | NM_004994 | ENSG00000100985 |
| IL8 | HGNC: 6025 | NM_000584 | ENSG00000169429 |
| VEGFA | HGNC: 12680 | NM_001025366 | ENSG00000112715 |
| APOE | HGNC: 613 | NM_000041 | ENSG00000130203 |
| SERPINA1 | HGNC: 8941 | NM_001002235 | ENSG00000197249 |
| SDC1 | HGNC: 10658 | NM_001006946 | ENSG00000115884 |

TABLE 3
Primer Probes of 10 analytes comprising bladder cancer diagnostic signature

| | NCBI | | FWD | | | | | | | Pro- |
|---|---|---|---|---|---|---|---|---|---|---|
| | Entrez | GenBank | or | Sequence (5→3) | | | | | GC | ducts |
| Genes | Gene | # | REV | [(SEQ ID NO)] | Length | Start | Stop | Tm | % | Length |
| SERPINE1 | 5054 | Nm_ | FWD | GCCAGTGGAAGACTCCCTTC (1) | 20 | 744 | 763 | 60.04 | 60 | 70 |
| (PAI-1) | | 000602.4 | REV | GTGCTGCCGTCTGATTTGTG (2) | 20 | 813 | 794 | 60.11 | 55 | |
| ANG | 283 | NM_ | FWD | TGGGCGTTTTGTTGTTGGTC (3) | 20 | 614 | 633 | 59.83 | 50 | 107 |
| | | 001145.4 | REV | GGCATCATAGTGCTGGGTCA (4) | 20 | 720 | 701 | 59.82 | 55 | |
| SDC1 | 6382 | NM_ | FWD | GCTCTGGCTCTGGCTGTG (5) | 18 | 278 | 295 | 60.44 | 66.67 | 148 |
| | | 002997.5 | REV | ATCTTGCAAAGCACCTGCAC (6) | 20 | 425 | 406 | 59.68 | 50 | |
| SERPINA1 | 5265 | NM_ | FWD | GTACCCTCAACCAGCCAGAC (7) | 20 | 421 | 440 | 60.04 | 60 | 147 |
| (A1AT) | | 000295.5 | REV | TGTCCCCGAAGTTGACAGTG (8) | 20 | 567 | 548 | 59.89 | 55 | |
| MMP9 | 4318 | NM_ | FWD | CAGTCCACCCTTGTGCTCTT (9) | 20 | 92 | 111 | 59.89 | 55 | 120 |
| | | 004994.3 | REV | CGACTCTCCACGCATCTCTG (10) | 20 | 211 | 192 | 60.25 | 60 | |
| MMP10 | 4319 | NM_ | FWD | CCTGGGTTTTCCTCCAACCA (11) | 20 | 1168 | 1187 | 59.81 | 55 | 140 |
| | | 002425.3 | REV | GTCTAGGGAAGCCTTGCTCC (12) | 20 | 1307 | 1288 | 59.82 | 60 | |
| CA9 | 768 | NM_ | FWD | GCCTTTGAATGGGCGAGTGA (13) | 20 | 1169 | 1188 | 60.96 | 55 | 127 |
| (CAIX) | | 001216.3 | REV | GCCAAAAACCAGGGCTAGGA (14) | 20 | 1295 | 1276 | 60.25 | 55 | |
| APOE | 348 | NM_ | FWD | CGCTTTTGGGATTACCTGCG (15) | 20 | 217 | 236 | 59.9 | 55 | 112 |
| | | 000041.4 | REV | TCATGGTCTCGTCCATCAGC (16) | 20 | 328 | 309 | 59.54 | 55 | |
| VEGFA | 7422 | NM_ | FWD | GTCCTGGAGCGTTCCCTGTG (17) | 20 | 1519 | 1538 | 62.79 | 65 | 110 |
| | | 003376.6 | REV | CCTTGCAACGCGAGTCTGTG (18) | 20 | 1628 | 1609 | 62.16 | 60 | |
| CXCL8 | 3576 | NM_ | FWD | CACTGCGCCAACACAGAAAT (19) | 20 | 268 | 287 | 59.69 | 50 | 121 |
| (IL8) | | 000584.4 | REV | ATGAATTCTCAGCCCTCTTCAA (20) | 22 | 388 | 367 | 57.42 | 40.91 | |

A prognostic method for bladder cancer can comprise:
(a) subjecting a sample comprising bladder cancer cells obtained from a patient to quantitative analysis of the expression level of the RNA transcript of at least one gene selected from the group consisting of ANG, A1AT, APOE, CA9, IL8, MMP9, MMP10, PAI-1, SDC1 and VEFGA, or their expression product, and (b) identifying the patient as likely to have a decreased likelihood of long-term survival without bladder cancer recurrence if the normalized expression levels of the gene or genes, or their products, are elevated above a defined expression threshold.

A kit may comprise one or more of (1) extraction buffer/reagents and protocol; (2) reverse transcription buffer/reagents and protocol; and (3) qPCR buffer/reagents and protocol suitable for performing any of the methods described herein. The kit may comprise an array, optionally a microarray, comprising cDNA transcripts consisting of ANG, A1AT, APOE, CA9, IL8, MMP9, MMP10, PAI-1, SDC1 and VEFGA.

An array can comprise a biomarker selected from the group consisting of ANG, A1AT, APOE, CA9, IL8, MMP9, MMP10, PAI-1, SDC1, VEFGA, and combinations thereof fixed to a substrate. The biomarkers can consist of ANG, A1AT, APOE, CA9, IL8, MMP9, MMP10, PAI-1, SDC1, and VEFGA. The biomarker can be an mRNA transcript. The biomarker can be a cDNA of the mRNA transcript. The biomarker can be a peptide.

A kit can comprise nucleic acid primers that specifically bind comprising a biomarker selected from the group consisting of ANG, A1AT, APOE, CA9, IL8, MMP9, MMP10, PAI-1, SDC1, VEFGA, and combinations. The biomarkers can consist of ANG, A1AT, APOE, CA9, IL8, MMP9, MMP10, PAI-1, SDC1, and VEFGA.

A kit can comprise antibodies that specifically bind comprising a biomarker selected from the group consisting of ANG, A1AT, APOE, CA9, IL8, MMP9, MMP10, PAI-1, SDC1, VEFGA, and combinations. The biomarkers can consist of ANG, A1AT, APOE, CA9, IL8, MMP9, MMP10, PAI-1, SDC1, and VEFGA.

The methods described herein can also be used in combination with other diagnostic techniques, invasive or non-invasive, as are known in the art at present.

Here, specificity is defined as the probability that a patient who did not have bladder cancer was assigned to the normal group, and the sensitivity is the probability that a patient who had bladder cancer was assigned to the disease group.

### Upper Tract Urothelial Cancers (UTUC)

Despite advances in technology, diagnosis of upper tract tumors continues to be challenging and often cytologies and/or biopsies are inconclusive or not performed due to the difficulty of reaching the lesion of concern. Consequently, the development of an accurate diagnostic assay that could be applied to non-invasively obtained urine samples would benefit both patients and health care systems.

Due to insufficient accuracy, urine-based assays currently have a limited role in the management of patients with upper tract urothelial cancers (UTUC). The application of a robust urine-based multiplex assay to aid in the diagnosis of UTUC has the potential to address this deficiency and to assist with accurate, non-invasive diagnosis.

The multiplex immunoassay described herein consisting of A1AT, APOE, ANG, CA9, IL8, MMP9, MMP10, PAI1, SDC1 and VEGFA showed an AUC of 0.897 (95% CI. 0.817-0.977) with an overall sensitivity of 93.5%, specificity of 75.6%, NPV 93.9% and PPV 74.4%. Sensitivity values of the diagnostic panel for high-grade UTUC, low-grade UTUC, non-invasive UTUC and invasive UTUC were 88.9%, 92.3%, 86.7% and 100%, respectively. Urinary cytology or selective ureteral washing/cytology was associated with an overall sensitivity of 58.3%, specificity of 100%, NPV 79.2% and PPV 100%. Sensitivity values of cytology for high-grade UTUC, low-grade UTUC, non-invasive UTUC and invasive UTUC were 50%, 100%, 80% and 42.9%, respectively.

Urinary levels of the biomarker panel consisting of A1AT, APOE, ANG, CA9, IL8, MMP9, MMP10, PAI1, SDC1, VEGFA, and combinations thereof, provided for the accurate discrimination of UTUC and controls non-tumor bearing individuals. The multiplex immunoassay test described herein can achieve the efficient and accurate detection of UTUC in a non-invasive patient setting. The multiplex immunoassay can use an array comprising a biomarker panel consisting of A1AT, APOE, ANG, CA9, IL8, MMP9, MMP10, PAI1, SDC1, VEGFA, and combinations thereof. The multiplex immunoassay can use an array comprising a biomarker panel consisting of A1AT, APOE, ANG, CA9, IL8, MMP9, MMP10, PAI1, SDC1, and VEGFA.

The protein biomarkers described herein (A1AT, APOE, ANG, CA9, IL8, MMP9, MMP10, PAI1, SDC1 VEGFA, and combinations thereof) can be found in the biological fluids inside a biomarker-positive cancer cell that is being shed or released in a fluid or biological sample under investigation, e.g., urine. Optionally, the sample may be blood, serum, plasma, urine, or a combination thereof. The sample may be urine. The biomarkers (A1AT, APOE, ANG, CA9, IL8, MMP9, MMP10, PAI1, SDC1 VEGFA, and combinations thereof) can also be found directly (i.e., cell-free) in the fluid or biological sample.

A method for detecting upper tract urothelial carcinoma (UTUC) biomarker can comprise (a) obtaining a biological sample from a subject; (b) contacting a biological sample obtained from a subject with a panel of binding agents, wherein said panel comprises binding agents that bind to, and form a complex, with proteins selected from the group consisting of ANG, A1AT, APOE, CA9, IL8, MMP9, MMP10, PAI-1, SDC1, VEFGA, and combinations thereof, and (c) detecting the presence and quantity of the protein-binding agent complexes that form in the biological sample.

The presence and quantity of biomarkers selected from the group consisting of ANG, A1AT, APOE, CA9, IL8, MMP9, MMP10, PAI-1, SDC1, VEFGA, and combinations thereof can be determined by an immunoassay. The protein biomarkers can consist of ANG, A1AT, APOE, CA9, IL8, MMP9, MMP10, PAI-1, SDC1, and VEFGA. As used herein, an “assay” or a diagnostic assay can be of any type applied in the field of diagnostics. Preferred detection methods comprise immunoassays in various formats such as for instance radioimmunoassays, chemiluminescence- and fluorescence-immunoassays, Enzyme-linked immunoassays (ELISA), Luminex-based bead arrays, protein microarray assays, assays suitable for point-of-care testing and rapid test formats such as for instance immune-chromatographic strip tests. Such an assay may be based on the binding of an analyte to be detected to one or more capture probes with a certain affinity. As used herein, an immunoassay is a biochemical test that measures the presence or concentration of a macromolecule/polypeptide in a solution through the use of an antibody or immunoglobulin. According to the invention, the antibodies may be monoclonal as well as polyclonal antibodies. Thus, at least one antibody is a monoclonal or polyclonal antibody.

The immunoassay can be selected from the group consisting of Luminescence immunoassay (LIA), radioimmunoassay (RIA), chemiluminescence- and fluorescence-immunoassay, enzyme immunoassay (EIA), Enzyme-linked immunoassay (ELISA), sandwich immunoassay, luminescence-based bead array, or a combination thereof. Immunoassay technology is described in the art, for example, Darwish Int J Biomed Sci (2006) 2(3): 217-235.

The proteins selected from the group consisting of ANG, A1AT, APOE, CA9, IL8, MMP9, MMP10, PAI-1, SDC1, VEFGA, and combinations thereof can be fixed to a substrate. The substrate can be a microplate or an array. The substrate can be an array. The biomarkers can consist of ANG, A1AT, APOE, CA9, IL8, MMP9, MMP10, PAI-1, SDC1, and VEFGA. An array can comprise antibodies that specifically bind to biomarker selected from the group consisting of ANG, A1AT, APOE, CA9, IL8, MMP9, MMP10, PAI-1, SDC1, VEFGA, and combinations thereof fixed to a substrate.

### Machine Learning

### Classification Systems

The invention relates to, among other things, characterizing biomarkers based on quantitative data on the expression level of a RNA transcript, preferably quantitative data on expression level of a RNA transcript from a tissue sample. The quantitative data on the expression level of a RNA transcript data sets may be propriety or accessed from publicly available databases. This data can be used to train machine learning systems to produce a classification on the diagnosis of cancer, optionally bladder cancer, and/or prognosis on the survival rate of subjects with cancer, optionally bladder cancer.

The classification systems used herein may include computer executable software, firmware, hardware, or combinations thereof. For example, the classification systems may include reference to a processor and supporting data storage. Further, the classification systems may be implemented across multiple devices or other components local or remote to one another. The classification systems may be implemented in a centralized system, or as a distributed system for additional scalability. Moreover, any reference to software may include non-transitory computer readable media that when executed on a computer, causes the computer to perform a series of steps.

The classification systems described herein may include data storage such as network accessible storage, local storage, remote storage (e.g., “cloud”), or a combination thereof. Data storage may utilize a redundant array of inexpensive disks (“RAID”), tape, disk, a storage area network (“SAN”), an internet small computer systems interface (“iSCSI”) SAN, a Fibre Channel SAN, a common Internet File System (“CIFS”), network attached storage (“NAS”), a network file system (“NFS”), or other computer accessible storage. The data storage may be a database, such as an Oracle database, a Microsoft SQL Server database, a DB2 database, a MySQL database, a Sybase database, an object oriented database, a hierarchical database, Cloud-based database, public database, or other database. Data storage may utilize flat file structures for storage of data.

In the first step, a classifier is used to describe a pre-determined set of data. This is the “learning step” and is carried out on “training” data.

The training database is a computer-implemented store of data reflecting a plurality of RNA expression level(s) data for a plurality of peptides association with a classification with respect to diagnostic and/or prognostic characterization of the biomarker levels. The RNA expression level(s) data may comprise experimental RNA expression level(s) data, predicted RNA expression level(s) data, or a combination thereof. The format of the stored data may be as a flat file, database, table, or any other retrievable data storage format known in the art. The test data may be stored as a plurality of vectors, each vector corresponding to an individual peptide, each vector including a plurality of RNA expression level(s) data measures for a plurality of experimental RNA expression level(s) data together with a classification with respect to antigenicity characterization of the peptide. The vector may further comprise retention time data measures for a plurality of experimental peptide retention data together with a classification with respect to the diagnostic and/or prognostic characterization of the biomarker levels. Typically, each vector contains an entry for each RNA expression level(s) data measure in the plurality of RNA expression level(s) data measures. The entry may further comprise retention time data. The training database may be linked to a network, such as the internet, such that its contents may be retrieved remotely by authorized entities (e.g., human users or computer programs). Alternately, the training database may be located in a network-isolated computer. Further, the training database may be Cloud-based, including proprietary and public databases containing RNA expression level(s) data (e.g., experimental, predicted, and combinations thereof) for biomarkers useful in immunooncology methods.

In the second step, which is optional, the classifier is applied in a “validation” database and various measures of accuracy, including sensitivity and specificity, are observed. In an exemplary embodiment, only a portion of the training database is used for the learning step, and the remaining portion of the training database is used as the validation database. In the third step, RNA expression level(s) data measures from a subject are submitted to the classification system, which outputs a calculated classification (e.g., diagnostic and/or prognostic characterization of the biomarker levels) for the subject. Additionally, other diagnostic data may also be used.

There are many possible classifiers that could be used on the data. Machine and deep learning classifiers include but are not limited to AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof, may be used to classify the data. See e.g., Han & Kamber (2006) Chapter 6, Data Mining, Concepts and Techniques, 2nd Ed. Elsevier: Amsterdam. As described herein, any classifier or combination of classifiers (e.g., ensemble) may be used in a classification system. As discussed herein, the data may be used to train a classifier.

Further a feature selection algorithm may be used in the machine learning application. For example, a feature selection algorithm may be used, including but not limited to Wrapper methods (forward, backward, and stepwise selection), Filter methods (ANOVA, Pearson correlation, variance thresholding), and Embedded methods (Lasso, Ridge, Decision Tree).

### Classification Trees

A classification tree is an easily interpretable classifier with built in feature selection. A classification tree recursively splits the data space in such a way so as to maximize the proportion of observations from one class in each subspace.

The process of recursively splitting the data space creates a binary tree with a condition that is tested at each vertex. A new observation is classified by following the branches of the tree until a leaf is reached. At each leaf, a probability is assigned to the observation that it belongs to a given class. The class with the highest probability is the one to which the new observation is classified.

Classification trees are essentially a decision tree whose attributes are framed in the language of statistics. They are highly flexible but very noisy (the variance of the error is large compared to other methods).

Tools for implementing classification tree are available, by way of non-limiting example, for the statistical software computing language and environment, R. For example, the R package “tree,” version 1.0-28, includes tools for creating, processing and utilizing classification trees. Examples of Classification Trees include but are not limited to Random Forest. See also Kaminski et al. (2017) “A framework for sensitivity analysis of decision trees.” Central European Journal of Operations Research. 26(1): 135-159; Karimi & Hamilton (2011) “Generation and Interpretation of Temporal Decision Rules”, International Journal of Computer Information Systems and Industrial Management Applications, Volume 3.

### Random Forests

Classification trees are typically noisy. Random forests attempt to reduce this noise by taking the average of many trees. The result is a classifier whose error has reduced variance compared to a classification tree. Methods of building a Random Forest classifier, including software, are known in the art. Prinzie & Poel (2007) “Random Multiclass Classification: Generalizing Random Forests to Random MNL and Random NB”. Database and Expert Systems Applications. Lecture Notes in Computer Science. 4653; Denisko & Hoffman (2018) “Classification and interaction in random forests”. PNAS 115(8): 1690-1692.

To classify a new observation using the random forest, classify the new observation using each classification tree in the random forest. The class to which the new observation is classified most often amongst the classification trees is the class to which the random forest classifies the new observation. Random forests reduce many of the problems found in classification trees but at the trade off of interpretability.

Tools for implementing random forests as discussed herein are available, by way of non-limiting example, for the statistical software computing language and environment, R. For example, the R package “random Forest,” version 4.6-2, includes tools for creating, processing and utilizing random forests.

### AdaBoost (Adaptive Boosting)

AdaBoost provides a way to classify each of n subjects into two or more categories based on one k-dimensional vector (called a k-tuple) of measurements per subject. AdaBoost takes a series of “weak” classifiers that have poor, though better than random, predictive performance and combines them to create a superior classifier. The weak classifiers that AdaBoost uses are classification and regression trees (CARTs). CARTs recursively partition the dataspace into regions in which all new observations that lie within that region are assigned a certain category label. AdaBoost builds a series of CARTs based on weighted versions of the dataset whose weights depend on the performance of the classifier at the previous iteration. Han & Kamber (2006) Data Mining, Concepts and Techniques, 2^{nd} Ed. Elsevier: Amsterdam, the content of which is incorporated by reference in its entirety. AdaBoost technically works only when there are two categories to which the observation can belong. For g>2 categories, (g/2) models must be created that classify observations as belonging to a group of not. The results from these models can then be combined to predict the group membership of the particular observation. Predictive performance in this context is defined as the proportion of observations misclassified.

### Convolutional Neural Network

Convolutional Neural Network (CNN or ConvNet) is a class of deep, feed-forward artificial neural networks, most commonly applied to analyzing visual imagery. CNNs use a variation of multilayer perceptrons designed to require minimal preprocessing. They are also known as shift invariant or space invariant artificial neural networks (SIANN), based on their shared-weights architecture and translation invariance characteristics. Convolutional networks were inspired by biological processes in that the connectivity pattern between neurons resembles the organization of the animal visual cortex. Individual cortical neurons respond to stimuli only in a restricted region of the visual field known as the receptive field. The receptive fields of different neurons partially overlap such that they cover the entire visual field. CNNs use relatively little pre-processing compared to other image classification algorithms. This means that the network learns the filters that in traditional algorithms were hand-engineered. This independence from prior knowledge and human effort in feature design is a major advantage. LeCun and Bengio (1995) “Convolutional networks for images, speech, and time-series,” in Arbib (Ed.), The Handbook of Brain Theory and Neural Networks, MIT Press. Fully convolutional indicates that the neural network is composed of convolutional layers without any fully-connected layers or MLP usually found at the end of the network. Convolutional Neural Network is an example of Deep learning.

### Support Vector Machines

Support vector machines (SVMs) are recognized in the art. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction. Vapnik (1998) Statistical Learning Theory; Vapnik “An overview of statistical learning theory” IEEE Transactions on Neural Networks 10(5): 988-999 (1999). Any new observation is then classified as belonging to any one of the categories of interest, based where the observation lies in relation to the hyperplane. When more than two categories are considered, the process is carried out pairwise for all of the categories and those results combined to create a rule to discriminate between all the categories.

A kernel function known as the Gaussian Radial Basis Function (RBF) can be used. Vapnik, 1998. The RBF is often used when no a priori knowledge is available with which to choose from a number of other defined kernel functions such as the polynomial or sigmoid kernels. Han et al. Data Mining: Concepts and Techniques Morgan Kaufman 3^{rd} Ed. (2012). The RBF projects the original space into a new space of infinite dimension. A discussion of this subject and its implementation in the R statistical language can be found in Karatzoglou et al. “Support Vector Machines in R” Journal of Statistical Software 15(9) (2006), the content of which is incorporated by reference in its entirety. All SVM statistical computations described herein were performed using the statistical software programming language and environment R 2.10.0. SVMs were fitted using the ksvm( ) function in the kernlab package. Cristianini, N., & Shawe-Taylor, J. (2000). An Introduction to Support Vector Machines and Other Kernel-based Learning Methods. Cambridge: Cambridge University Press provides some notation for support vector machines, as well as an overview of the method by which they discriminate between observations from multiple groups.

Other suitable Kernel functions include, but are not limited to, linear kernels, radial basis Kernels, polynomial Kernels, uniform Kernels, triangle Kernels, Epanechnikov Kernels, quartic (biweight) Kernels, tricube (triweight) Kernels, and cosine Kernels.

Support vector machines are one out of many possible classifiers that could be used on the data.

By way of non-limiting example, and as discussed below, other methods such as naive Bayes classifiers, classification trees, k-nearest neighbor classifiers, etc. may be used on the same data used to train and verify the support vector machine.

### Naïve Bayes Classifier

The set of Bayes Classifiers are a set of classifiers based on Bayes' Theorem. See, e.g., Joyce (2003), Zalta, Edward N. (ed.), “Bayes' Theorem”, The Stanford Encyclopedia of Philosophy (Spring 2019 Ed.), Metaphysics Research Lab, Stanford University.

All classifiers of this type seek to find the probability that an observation belongs to a class given the data for that observation. The class with the highest probability is the one to which each new observation is assigned. Theoretically, Bayes classifiers have the lowest error rates amongst the set of classifiers. In practice, this does not always occur due to violations of the assumptions made about the data when applying a Bayes classifier.

The naïve Bayes classifier is one example of a Bayes classifier. It simplifies the calculations of the probabilities used in classification by making the assumption that each class is independent of the other classes given the data.

Naïve Bayes classifiers are used in many prominent anti-spam filters due to the ease of implantation and speed of classification but have the drawback that the assumptions required are rarely met in practice.

Tools for implementing naive Bayes classifiers as discussed herein are available for the statistical software computing language and environment, R. For example, the R package “e1071,” version 1.5-25, includes tools for creating, processing and utilizing naive Bayes classifiers.

### Neural Nets

One way to think of a neural net is as a weighted directed graph where the edges and their weights represent the influence each vertex has on the others to which it is connected. There are two parts to a neural net: the input layer (formed by the data) and the output layer (the values, in this case classes, to be predicted). Between the input layer and the output layer is a network of hidden vertices. There may be, depending on the way the neural net is designed, several vertices between the input layer and the output layer.

Neural nets are widely used in artificial intelligence and data mining but there is the danger that the models the neural nets produce will over fit the data (i.e., the model will fit the current data very well but will not fit future data well). Tools for implementing neural nets as discussed herein are available for the statistical software computing language and environment, R. For example, the R package “e1071,” version 1.5-25, includes tools for creating, processing and utilizing neural nets.

k-Nearest Neighbor Classifiers (KNN)

The nearest neighbor classifiers are a subset of memory-based classifiers. These are classifiers that have to “remember” what is in the training set in order to classify a new observation. Nearest neighbor classifiers do not require a model to be fit.

To create a k-nearest neighbor (knn) classifier, the following steps are taken:
1. Calculate the distance from the observation to be classified to each observation in the training set. The distance can be calculated using any valid metric, though Euclidian and Mahalanobis distances are often used.
2. Count the number of observations amongst the k nearest observations that belong to each group.
3. The group that has the highest count is the group to which the new observation is assigned.

The Mahalanobis distance is a metric that takes into account the covariance between variables in the observations.

Nearest neighbor algorithms have problems dealing with categorical data due to the requirement that a distance be calculated between two points but that can be overcome by defining a distance arbitrarily between any two groups. This class of algorithm is also sensitive to changes in scale and metric. With these issues in mind, nearest neighbor algorithms can be very powerful, especially in large data sets.

Tools for implementing k-nearest neighbor classifiers as discussed herein are available for the statistical software computing language and environment, R. For example, the R package “e1071,” version 1.5-25, includes tools for creating, processing and utilizing k-nearest neighbor classifiers.

### Training Data

In another aspect, methods described herein include training of about 75%, about 80%, about 85%, about 90%, or about 95% of the data in the library or database and testing the remaining percentage for a total of 100% data. In an aspect, from about 70% to about 90% of the data is trained and the remainder of about 10% to about 30% of the data is tested, from about 80% to about 95% of the data is trained and the remainder of about 5% to about 20% of the data is tested, or from about 90% of the data is trained and the remainder of about 10% of the data is tested.

In an aspect, the database or library contains data from the analysis of over about 500, about 1000, over about 1500, over about 2000, over about 2500, or over about 3000 tissue samples, preferably tumor tissue samples. In an aspect, tumor tissue and healthy tissue from the same individual were analyzed.

### Methods of Classifying Data Using Classification System(s)

The invention provides for methods of classifying data (test data, e.g., quantitative RNA expression data) obtained from an individual. These methods involve preparing or obtaining training data, as well as evaluating test data obtained from an individual (as compared to the training data), using one of the classification systems including at least one classifier as described above. Preferred classification systems use classifiers such as, but not limited to, support vector machines (SVM), AdaBoost, penalized logistic regression, naive Bayes classifiers, classification trees, k-nearest neighbor classifiers, Deep Learning classifiers, neural nets, random forests, Fully Convolutional Networks (FCN), Convolutional Neural Networks (CNN), and/or an ensemble thereof. Deep Learning classifiers are a more preferred classification system. The classification system outputs a classification of the peptide based on the test data, e.g., quantitative RNA expression data.

Particularly preferred for the present invention is an ensemble method used on a classification system, which combines multiple classifiers. For example, an ensemble method may include SVM, AdaBoost, penalized logistic regression, naive Bayes classifiers, classification trees, k-nearest neighbor classifiers, neural nets, Fully Convolutional Networks (FCN), Convolutional Neural Networks (CNN), Random Forests, Deep Learning, or any ensemble thereof, in order to make a prediction regarding peptide antigenicity (e.g., HLA peptide, antigenic peptide). The ensemble method was developed to take advantage of the benefits provided by each of the classifiers, and replicate measurements of each RNA expression level(s) data.

A method of classifying test data, the test data comprising quantitative RNA expression data for a subset of biomarkers comprising: (a) accessing an electronically stored set of training data vectors, each training data vector or k-tuple representing an individual biomarker and comprising RNA expression level(s) data for the respective biomarker for each replicate, the training data vector further comprising a classification with respect to diagnostic and/or prognostic characterization of each respective biomarker; (b) training an electronic representation of a classifier or an ensemble of classifiers as described herein using the electronically stored set of training data vectors; (c) receiving test data comprising a plurality of RNA expression level(s) data for the biomarker(s); (d) evaluating the test data using the electronic representation of the classifier and/or an ensemble of classifiers as described herein; and (e) outputting a classification of the peptide based on the evaluating step. The test data may further comprise other data from the subject, including but not limited to histological, metabolic data, signs, symptoms, or combinations thereof.

In another embodiment, the invention provides a method of classifying test data, the test data comprising quantitative RNA expression data comprising: (a) accessing an electronically stored set of training data vectors, each training data vector or k-tuple representing an individual human and comprising quantitative RNA expression data for the respective human for each replicate, the training data further comprising a classification with respect to diagnostic and/or prognostic value of each respective biomarker; (b) using the electronically stored set of training data vectors to build a classifier and/or ensemble of classifiers; (c) receiving test data comprising a plurality of quantitative RNA expression data for a human test subject; (d) evaluating the test data using the classifier(s); and (e) outputting a classification of the human test subject based on the evaluating step. Alternatively, all (or any combination of) the replicates may be averaged to produce a single value for each biomarker for each subject. Outputting in accordance with this invention includes displaying information regarding the classification of the human test subject in an electronic display in human-readable form.

The set of training vectors may comprise at least 20, 25, 30, 35, 50, 75, 100, 125, 150, or more vectors.

The test data may be any signs, symptoms, or other data measures such as possible histological data, metabolite data, patient demographics, tumor (cancer) characteristics, treatment, outcomes, or a combination thereof.

The data used to train a machine learning system, e.g., Deep Learning, may comprise data from tumors, including at least 5, 10, 15, 20, or 25 different indications, data from normal tissues, including at least about 5, 10, 15, 20, 25, 30, 35, 40, or 45 normal (tumor-free) tissues, or a combination thereof. In addition, the data used to train a machine learning system, e.g., Deep Learning, may comprise CID (Collision-induced dissociation) data, HCD (Higher-energy collisional dissociation) data, or a combination thereof.

It will be understood that the methods of classifying data may be used in any of the methods described herein. In particular, the methods of classifying data described herein may be used in methods for characterization of the biomarkers, e.g., ANG, A1AT, APOE, CA9, IL8, MMP9, MMP10, PAI-1, SDC1 and VEFGA, for use in immunooncology methods.

Particularly preferred for the present invention is an ensemble method used on a classification system, which combines multiple classifiers. For example, an ensemble method may include Support Vector Machine (SVM), AdaBoost, penalized logistic regression, naive Bayes classifiers, classification trees, k-nearest neighbor classifiers, neural nets, Deep Learning systems, Random Forests, or any combination thereof, in order to make a prediction regarding diagnostic and/or prognostic characterization of a biomarker, including a subset of biomarkers, e.g., ANG, A1AT, APOE, CA9, IL8, MMP9, MMP10, PAI-1, SDC1 and VEFGA. In addition, the ensemble may be used to make a prediction regarding the association of the subset of biomarkers (ANG, A1AT, APOE, CA9, IL8, MMP9, MMP10, PAI-1, SDC1 and VEFGA) with a type of cancer and an outcome for the patient. The ensemble approach takes advantage of the benefits provided by each of the classifiers, and replicate measurements of each biomarker(s) (ANG, A1AT, APOE, CA9, IL8, MMP9, MMP10, PAI-1, SDC1 and VEFGA).

### Computer-Implemented Methods

As used herein, the term “computer” is to be understood to include at least one hardware processor that uses at least one memory. The at least one memory may store a set of instructions. The instructions may be either permanently or temporarily stored in the memory or memories of the computer. The processor executes the instructions that are stored in the memory or memories in order to process data. The set of instructions may include various instructions that perform a particular task or tasks, such as those tasks described herein. Such a set of instructions for performing a particular task may be characterized as a program, software program, or simply software.

As noted above, the computer executes the instructions that are stored in the memory or memories to process data. This processing of data may be in response to commands by a user or users of the computer, in response to previous processing, in response to a request by another computer and/or any other input, for example.

The computer used to at least partially implement embodiments may be a general purpose computer. However, the computer may also utilize any of a wide variety of other technologies including a special purpose computer, a computer system including a microcomputer, mini-computer or mainframe for example, a programmed microprocessor, a micro-controller, a peripheral integrated circuit element, a CSIC (Customer Specific Integrated Circuit) or ASIC (Application Specific Integrated Circuit) or other integrated circuit, a logic circuit, a digital signal processor, a programmable logic device such as a FPGA, PLD, PLA or PAL, or any other device or arrangement of devices that is capable of implementing at least some of the steps of the processes of the invention.

It is appreciated that in order to practice the method of the invention, it is not necessary that the processors and/or the memories of the computer be physically located in the same geographical place. That is, each of the processors and the memories used by the computer may be located in geographically distinct locations and connected so as to communicate in any suitable manner. Additionally, it is appreciated that each of the processor and/or the memory may be composed of different physical pieces of equipment. Accordingly, it is not necessary that the processor be one single piece of equipment in one location and that the memory be another single piece of equipment in another location. That is, it is contemplated, for example, that the processor may be two or more pieces of equipment in two different physical locations. The two or more distinct pieces of equipment may be connected in any suitable manner, such as a network. Additionally, the memory may include two or more portions of memory in two or more physical locations.

Various technologies may be used to provide communication between the various computers, processors and/or memories, as well as to allow the processors and/or the memories of the invention to communicate with any other entity; e.g., so as to obtain further instructions or to access and use remote memory stores, for example. Such technologies used to provide such communication might include a network, the Internet, Intranet, Extranet, LAN, an Ethernet, or any client server system that provides communication, for example. Such communications technologies may use any suitable protocol such as TCP/IP, UDP, or OSI, for example.

Further, it is appreciated that the computer instructions or set of instructions used in the implementation and operation of the invention are in a suitable form such that a computer may read the instructions.

In some embodiments, a variety of user interfaces may be utilized to allow a human user to interface with the computer or machines that are used to at least partially implement the embodiment. A user interface may be in the form of a dialogue screen. A user interface may also include any of a mouse, touch screen, keyboard, voice reader, voice recognizer, dialogue screen, menu box, list, checkbox, toggle switch, a pushbutton or any other device that allows a user to receive information regarding the operation of the computer as it processes a set of instructions and/or provide the computer with information. Accordingly, a user interface is any device that provides communication between a user and a computer. The information provided by the user to the computer through the user interface may be in the form of a command, a selection of data, or some other input, for example.

It is also contemplated that a user interface of the invention might interact, e.g., convey and receive information, with another computer, rather than a human user. Accordingly, the other computer might be characterized as a user. Further, it is contemplated that a user interface utilized in the system and method of the invention may interact partially with another computer or computers, while also interacting partially with a human user.

### Nucleic Acid Assays

Nucleic acids, including naturally occurring nucleic acids, oligonucleotides, antisense oligonucleotides, and synthetic oligonucleotides that hybridize to the nucleic acid encoding biomarker polypeptides of the invention, are useful as agents to detect the presence of biomarkers of the invention in the biological samples of cancer patients or those at risk of cancer, preferably in the urine of bladder cancer patients or those at risk of bladder cancer. The present invention contemplates the use of nucleic acid sequences corresponding to the coding sequence of biomarkers of the invention and to the complementary sequence thereof, as well as sequences complementary to the biomarker transcript sequences occurring further upstream or downstream from the coding sequence (e.g., sequences contained in, or extending into, the 5′ and 3′ untranslated regions) for use as agents for detecting the expression of biomarkers of the invention in biological samples of cancer patients, or those at risk of cancer, preferably in the urine of bladder cancer patients or those at risk of bladder cancer.

The preferred oligonucleotides for detecting the presence of biomarkers of the invention in biological samples are those that are complementary to at least part of the cDNA sequence encoding the biomarker. These complementary sequences are also known in the art as “antisense” sequences. These oligonucleotides may be oligoribonucleotides or oligodeoxyribonucleotides. In addition, oligonucleotides may be natural oligomers composed of the biologically significant nucleotides, i.e., A (adenine), dA (deoxyadenine), G (guanine), dG (deoxyguanine), C (cytosine), dC (deoxycytosine), T (thymine), and U (uracil), or modified oligonucleotide species, substituting, for example, a methyl group or a sulfur atom for a phosphate oxygen in the inter-nucleotide phosphodiester linkage. Additionally, these nucleotides themselves, and/or the ribose moieties, may be modified.

The oligonucleotides may be synthesized chemically, using any of the known chemical oligonucleotide synthesis methods known in the art. Ausubel, et al. [Ed.] Short Protocols in Molecular Biology (5^{th} Ed.) (2002). For example, the oligonucleotides can be prepared by using any of the commercially available, automated nucleic acid synthesizers. Alternatively, the oligonucleotides may be created by standard recombinant DNA techniques, for example, inducing transcription of the noncoding strand. The DNA sequence encoding the biomarker may be inverted in a recombinant DNA system, e.g., inserted in reverse orientation downstream of a suitable promoter, such that the noncoding strand now is transcribed.

Although any length oligonucleotide may be utilized to hybridize to a nucleic acid encoding a biomarker polypeptide, oligonucleotides typically within the range of 8-100 nucleotides are preferred. Most preferable oligonucleotides for use in detecting biomarkers in urine samples are those within the range of 15-50 nucleotides.

The oligonucleotide selected for hybridizing to the biomarker nucleic acid molecule, whether synthesized chemically or by recombinant DNA technology, is then isolated and purified using standard techniques and then preferably labeled (e.g., with ³⁵S or ³²P) using standard labeling protocols.

Oligonucleotide pairs can be used in polymerase chain reactions (PCR) to detect the expression of the biomarker in biological samples, optionally quantitative PCR methods. The oligonucleotide pairs include a forward primer and a reverse primer.

The presence of biomarkers in a sample from a patient may be determined by nucleic acid hybridization, such as, but not limited to, Northern blot analysis, dot blotting, Southern blot analysis, fluorescence in situ hybridization (FISH), PCR and RNA sequencing. Chromatography, preferably HPLC, and other known assays may also be used to determine messenger RNA levels of biomarkers in a sample.

Nucleic acid molecules encoding a biomarker described herein can be found in the biological fluids inside a biomarker-positive cancer cell that is being shed or released in a fluid or biological sample under investigation, e.g., urine. Optionally, the sample may be blood, serum, plasma, urine, or a combination thereof. The sample may be urine. Nucleic acids encoding biomarkers can also be found directly (i.e., cell-free) in the fluid or biological sample.

The nucleic acids used as agents for detecting biomarkers described herein in biological samples of patients, can be labeled. The nucleic acids can be labeled with a radioactive label, a fluorescent label, an enzyme, a chemiluminescent tag, a colorimetric tag, or a combination thereof. The mRNA transcripts of biomarkers consisting of ANG, A1AT, APOE, CA9, IL8, MMP9, MMP10, PAI-1, SDC1 and VEFGA fixed to a substrate in a microarray. A microarray may comprise cDNA transcripts of ANG, A1AT, APOE, CA9, IL8, MMP9, MMP10, PAI-1, SDC1 and VEFGA fixed to a substrate in a microarray. An array can comprise a biomarker selected from the group consisting of ANG, A1AT, APOE, CA9, IL8, MMP9, MMP10, PAI-1, SDC1, VEFGA, and combinations thereof fixed to a substrate. The biomarkers can consist of ANG, A1AT, APOE, CA9, IL8, MMP9, MMP10, PAI-1, SDC1, VEFGA. The biomarker on the array can be an mRNA transcript. The biomarker on the array can be a cDNA of the mRNA transcript. The biomarker on the array can be a peptide.

The detection methods described herein can produce an output (e.g., readout or signal) with information concerning the outcomes of bladder cancer subjects. For example, the output may be qualitative (e.g., “responder” or “non-responder”), or quantitative (e.g., a concentration such as nanograms per milliliter).

### Representative Terms

Unless otherwise indicated, all terms used herein have the same meaning as they would to one skilled in the art.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

“AdaBoost,” as used herein, refers broadly to a bagging method that iteratively fits CARTs re-weighting observations by the errors made at the previous iteration.

“Cancer” and “cancerous,” as used herein, refers broadly to the physiological condition in mammals that is typically characterized by unregulated cell growth. Examples of cancer include but are not limited to, bladder cancer, colon cancer, lung cancer, prostate cancer, hepatocellular cancer, gastric cancer, pancreatic cancer, cervical cancer, ovarian cancer, liver cancer, breast cancer, cancer of the urinary tract, thyroid cancer, renal cancer, melanoma, and brain cancer.

“Classifier,” as used herein, refers broadly to a machine learning algorithm such as support vector machine(s), AdaBoost classifier(s), penalized logistic regression, elastic nets, regression tree system(s), gradient tree boosting system(s), naive Bayes classifier(s), neural nets, Bayesian neural nets, k-nearest neighbor classifier(s), Deep Learning systems, and random forests. This invention contemplates methods using any of the listed classifiers, as well as use of more than one of the classifiers in combination.

“Classification and Regression Trees (CART),” as used herein, refers broadly to a method to create decision trees based on recursively partitioning a data space so as to optimize some metric, usually model performance.

“Classification system,” as used herein, refers broadly to a machine learning system executing at least one classifier.

“Differentially expressed gene,” “differential gene expression”, as used herein, refer broadly to a gene whose expression is activated toa higher or lower level in a subject suffering from a disease, specifically cancer, such as bladder cancer, relative to its expression in a normal or control subject. The terms also include genes whose expression is activated to a higher or lower level at different stages of the same disease. It is also understood that a differentially expressed gene may be either activated or inhibited at the nucleic acid level or protein level or may be subject to alternative splicing to result in a different polypeptide product. Such differences may be evidenced by a change in mRNA levels, surface expression, secretion, or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about six-fold, most preferably at least about ten-fold difference between the expression of a given gene in normal and diseased subjects, or in various stages of disease development in a diseased subject.

“Elastic Net,” as used herein, refers broadly to a method for performing linear regression with a constraint comprised of a linear combination of the L1 norm and L2 norm of the vector of regression coefficients.

“Expression threshold,” and “defined expression threshold,” can be used interchangeably and refer broadly to the level of a gene or gene product in question above which the gene or gene product serves as a predictive marker for patient survival without cancer recurrence. The threshold is defined experimentally from clinical studies such as those described in the Example below. The expression threshold can be selected either for maximum sensitivity, or for maximum selectivity, or for minimum error. The determination of the expression threshold for any situation is well within the knowledge of those skilled in the art.

“False Positive (FP)” and “False Positive Identification,” as used herein, refers broadly to an error in which the algorithm test result indicates the presence of a disease when the disease is actually absent.

“False Negative (FN),” as used herein, refers broadly to an error in which the algorithm test result indicates the absence of a disease when the disease is actually present.

“Gene amplification,” as used herein, refers broadly to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

“HLA peptide,” as used herein, refers broadly to an antigenic peptide that is bound in a peptide-MHC complex and presented to a T-cell. HLA peptides are antigenic peptides.

“LASSO,” as used herein, refers broadly to a method for performing linear regression with a constraint on the L1 norm of the vector of regression coefficients.

“L1 Norm,” as used herein, is the sum of the absolute values of the elements of a vector.

“L2 Norm,” as used herein, is the square root of the sum of the squares of the elements of a vector.

“Long-term survival,” as used herein, refers broadly to survival for at least 3 years, more preferably for at least 8 years, most preferably for at least 10 years following surgery or other treatment.

“Mammal,” as used herein, refers broadly to any and all warm-blooded vertebrate animals of the class Mammalia, characterized by a covering of hair on the skin and, in the female, milk-producing mammary glands for nourishing the young. Mammals include, but are not limited to, humans, domestic and farm animals, and zoo, sports, or pet animals. Examples of mammals include but are not limited to alpacas, armadillos, capybaras, cats, camels, chimpanzees, chinchillas, cattle, dogs, gerbils, goats, gorillas, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C. Similarly, the term “subject” or “patient” includes both human and veterinary subjects and/or patients.

“Negative Predictive Value (NPV),” as used herein, is the number of true negatives (TN) divided by the number of true negatives (TN) plus the number of false negatives (FP), TP/(TN+FN).

“Neural Net,” as used herein, refers broadly to a classification method that chains together perceptron-like objects to create a classifier.

“Performance score,” as used herein, refers broadly to the distances between predicted values and actual values in the training data. This is expressed as a number between 0-100%, with higher values indicating the predicted value is closer to the real value. Typically, a higher score means the model performs better.

“Polynucleotide,” as used herein, refers broadly to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions maybe from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

“Positive Predictive Value (PPV),” is the number of true positives (TP) divided by the number of true positives (TP) plus the number of false positives (FP), TP/(TP+FP).

“Prediction,” as used herein, refers broadly to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention can be used clinically to make treatment decisions by choosing the most appropriate treatment modalities for any particular patient. The predictive methods of the present invention are valuable tools in predicting if a patient is likely to respond favorably to a treatment regimen, such as surgical intervention, chemotherapy, immunotherapy, radiation therapy or any combination of these therapies, or whether long-term survival of the patient, following surgery and/or termination of chemotherapy or other treatment modalities is likely.

“Prognosis,” as used herein, refers broadly to the prediction of the likelihood of cancer-attributable death or progression, including recurrence, metastatic spread, and drug resistance, of a neoplastic disease, for example, bladder cancer.

“Random Forest,” as used herein, refers broadly to a bagging method that fits CARTs based on samples from the dataset that the model is trained on.

“Ridge Regression,” as used herein, refers broadly to a method for performing linear regression with a constraint on the L2 norm of the vector of regression coefficients.

“Sample,” “biological sample,” refer broadly to a type of material known to or suspected of expressing or containing a biomarker of cancer, such as tumor. The test sample can be used directly as obtained from the source or following a pretreatment to modify the character of the sample. The sample can be derived from any biological source, such as tissues or extracts, including cells (e.g., tumor cells) and physiological fluids, such as, for example, whole blood, plasma, serum, peritoneal fluid, ascites, and the like. The sample can be obtained from animals, preferably mammals, most preferably humans. The sample can be pretreated by any method and/or can be prepared in any convenient medium that does not interfere with the assay. The sample can be treated prior to use, such as preparing plasma from blood, diluting viscous fluids, applying one or more protease inhibitors to samples such as urine, and the like. Sample treatment can involve filtration, distillation, extraction, concentration, inactivation of interfering components, the addition of reagents.

“Standard of Deviation (SD),” as used herein, is the spread in individual data points (i.e., in a replicate group) to reflect the uncertainty of a single measurement.

“Subject” and “patient,” are used interchangeably and refer broadly to a mammal, which may be afflicted with cancer such as bladder cancer. The subject may be male or female.

“Subset,” as used herein, refer broadly to a proper subset and “superset” is a proper superset.

“Training Set,” as used herein, is the set of samples that are used to train and develop a machine learning system, such as an algorithm used in the method and systems described herein.

“True Negative (TN),” as used herein, is the algorithm test result indicates that a peptide is not an antigenic when the peptide is actually antigenic.

“True Positive (TP),” as used herein, is the algorithm test result indicates that a peptide is antigenic when the peptide is actually antigenic.

“Tumor,” as used herein, refers broadly to all neoplastic cell growth and proliferation, whether malignant or benign, and all pre-cancerous and cancerous cells and tissues.

“Validation Set,” as used herein, refers broadly to the set of samples that are blinded and used to confirm the functionality of the algorithm used in the method and systems described herein. This is also known as the Blind Set.

### EXAMPLES

### Example 1

### Diagnostic Bladder Cancer Signature

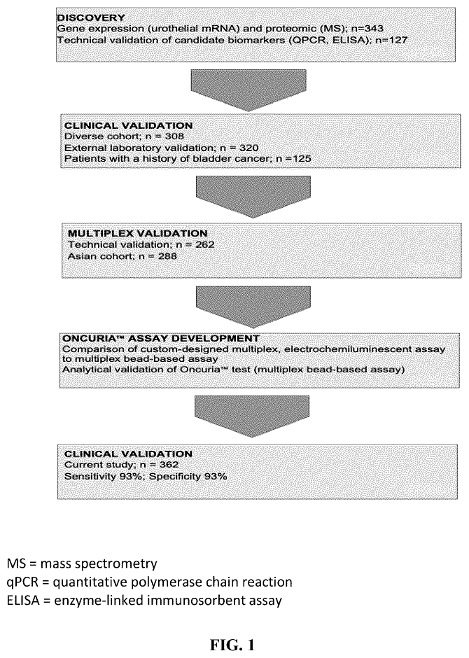

The methodological approach the inventors deployed to discover and validate a diagnostic bladder cancer signature is depicted in FIG. 1. The inventors developed this approach to test numerous possible choices until one possibly arrived at a successful result, and the prior art gave either no indication of which parameters were critical or no direction as to which of many possible choices is likely to be successful. Briefly, two complementary techniques were applied to profile urine samples from patients with or without bladder cancer; gene expression (mRNA) of shed urothelia (Rosser et al. Cancer Epidemiol Biomarkers Prev. (2009) 18(2): 444-53; Urquidi et al. Cancer Epidemiol Biomarkers Prev. (2012) 21(12): 2149-58), and glycoproteomics profiling of urine supernatant (Kreunin et al. J Proteome Res. (2007) 6(7): 2631-9; Yang et al. Clin Cancer Res. (2011) 17(10): 3349-59).

To derive a diagnostic optimal multi-gene diagnostic signature from the microarray data, the LoGo feature selection algorithm was applied. Sun et al. IEEE Trans Pattern Anal Mach Intell. (2010) 32(9): 1610-1626; Sun et al. Prostate (2009) 69(10): 1119-1127. To avoid possible overfitting of computational models to training data, the leave-one-out cross validation (LOOCV) method was used to estimate classifier parameters and prediction performance. Goodison et al. Bioanalysis (2010) 2(5): 855-862. A receiver operating characteristic (ROC) curve (van Vliet et al. BMC Genomics (2008) 9: 375) obtained by varying a decision threshold was used to provide a direct view of how a prediction model performed at different sensitivity and specificity levels. Scatter plots were created to illustrate relative prediction scores and significance between groups was evaluated using t-tests.

Then, using advanced bioinformatics, the two datasets were combined, and a bladder cancer-associated signature comprised of 14 candidate biomarkers was identified. [ANG, A1AT, APOE, CA9, CCL18, CD44, IL8, MMP9, MMP10, OPN, PAI-1, PTX, SDC1 and VEFGA] This “signature” was composed of both mRNA and glycoproteins. The inventors validated these 14 biomarkers at the protein level with 14 individual commercial ELISA kits. The potential clinical utility of the candidate 14 protein biomarkers was monitored in voided urine samples from an independent cohort of 127 patients (64 with bladder cancer). Of the 14 biomarkers, we confirmed that 10 were significantly different between cancers and controls. Goodison et al. PLoS One (2012) 7(10: e47469.

These 10 protein biomarkers included angiogenin, ANG; apolipoprotein E, APOE; alpha-1 antitrypsin, A1AT; carbonic anhydrase 9, CA9; interleukin 8, IL8; matrix metallopeptidase 9, MMP9; matrix metallopeptidase 10, MMP10; plasminogen activator inhibitor 1, PAI1; syndecan 1, SDC1 and vascular endothelial growth factor A, VEGFA, achieving a diagnostic sensitivity of 92% at a specificity of 97% when combined using logistic regression. Appreciating that benign conditions can adversely affect the performance of urinary biomarkers, the bladder cancer-associated signature was confirmed in an independent cohort comprised of 102 bladder cancer patients and 206 controls with a sensitivity of 74% at a specificity of 90%. The controls included patients with diverse benign conditions such as urinary tract infection, hematuria with no cancer, kidney stones, moderate to severe voiding symptoms and erectile dysfunction. Rosser et al. J. Urol. (2013) 190(6): 2257-62.

Subsequently, the bladder cancer-associated signature was validated by an independent laboratory in a cohort comprised of 183 bladder cancer patients and 137 controls with a sensitivity of 79% at a specificity of 79%. Chen et al. Cancer Epidemiol Biomarkers Prev. (2014) 23(9): 1804-12. Next, the “signature” was also confirmed to perform equally well for the detection of recurrent bladder cancer in a cohort of 125 patients (53 recurrent cancers and 72 non-tumor recurrence) on disease surveillance, outperforming both UroVysion Bladder Cancer Kit (Abbott) and VUC in this context, sensitivity and specificity of 79% and 88%, 42% and 94% and 33% and 90%, respectively. Rosser et al. Cancer Epidemiol Biomarkers Prev. (2014) 23(7): 1340-5. The analysis of cumulative data from over 1,100 patients confirmed the diagnostic power of the multi-factor protein “signature” over individual biomarkers, regardless of histological grade or disease stage of tumors (Masuda et al. Oncotarget (2018) 9: 7101-11), prompting us to develop a multiplex immunoassay. Prototypes of a multiplex immunoassay were tested in two large independent cohorts. In a US cohort of 200 patients (100 with bladder cancer), the immunoassay achieved a diagnostic sensitivity of 80% at a specificity of 81% (Shimizu et al. J Transl Med (2016) 14:31), and in a Japanese cohort of 278 patients (211 with bladder cancer), an optimized iteration of the multiplex immunoassay achieved a diagnostic sensitivity of 85% at a specificity of 81% (Goodison et al. J. Transl Med. (2016) 14(1): 287). Furthermore in 2019, we have tested and compared the performance of the multiplex immunoassay on two different technology platforms (Furuya et al. Diagnostics (2019) 9(4)).

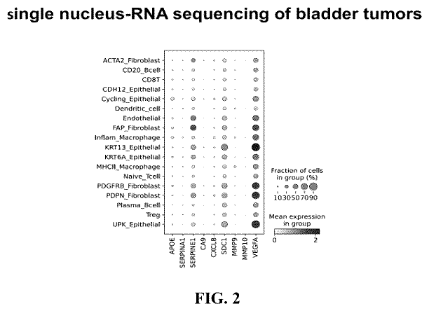

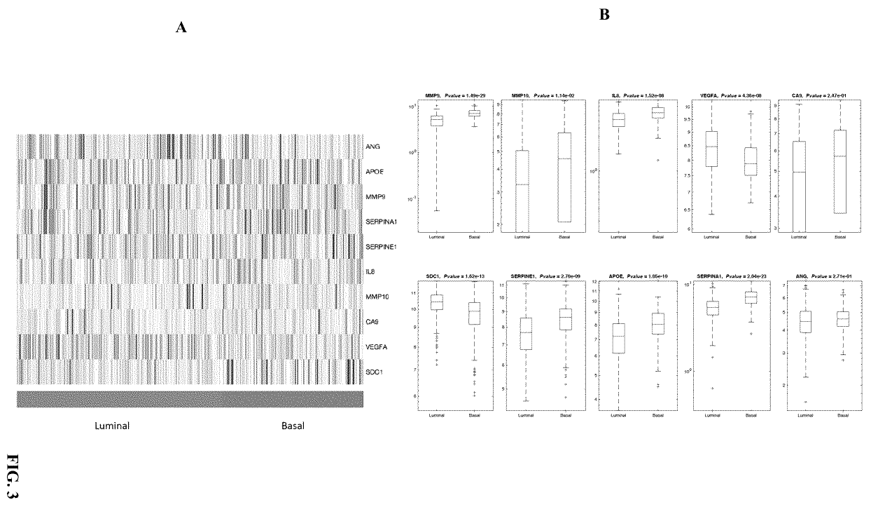

The inventors performed gene expression array, however, using more contemporary methods such as single cell RNA sequencing, and noted that the presence of 9 of the 10 analytes over expressed in human bladder tumors (n=25; Gouin et al. Nat Commun. (2021) 12(1): 4906) (FIG. 2). Several immunohistochemical staining studies demonstrated that these 10 analytes at the protein level were present in human tumors and could be linked to tumor grade, tumor stage and clinical outcomes.

Analytical validation of the test has assessed selectivity, sensitivity, specificity, accuracy, linearity, dynamic range, and detection threshold, using voided urine as the test matrix (Huang et al. Cancer Epidemiol Biomarkers Prev. (2016) 25(9): 1361-6. Lower and upper limits of quantification (LLOQ and ULOQ), antigen cross-reactivity, and the effect of potential interference of the assay by matrix substances has been defined.

A small clinical validation study consisting of a cohort of 362 patients (46 with bladder cancer) was performed. The median age of bladder cancer subjects was 69 years (range 38-87 years), 76.1% were men and 67.4% were Caucasian. Of the 46 bladder cancer cases, 61.4% were classified NMIBC; stages Ta, Tis, T1), and 38.6% were MIBC; stage ≥T2, 19.6% cases were reported as low-grade cancer and 80.4% cases as high-grade (Hirasawa et al. J. Transl Med. (2021) 19(1): 141).

Transcriptional and survival data from bladder cancer patients from The Cancer Genome Atlas (TCGA) were analyzed for the 10 analytes; ANG, A1AT, APOE, CA9, IL8, MMP9, MMP10, PAI-1, SDC1 and VEFGA as it relates to the bladder cancer molecular subtypes. A survival analysis was carried out to explore the individual as well as prognostic significance of the 10 analytes. Eight of the 10 analytes were significantly associated with the reported bladder cancer molecular subtypes (FIG. 3).

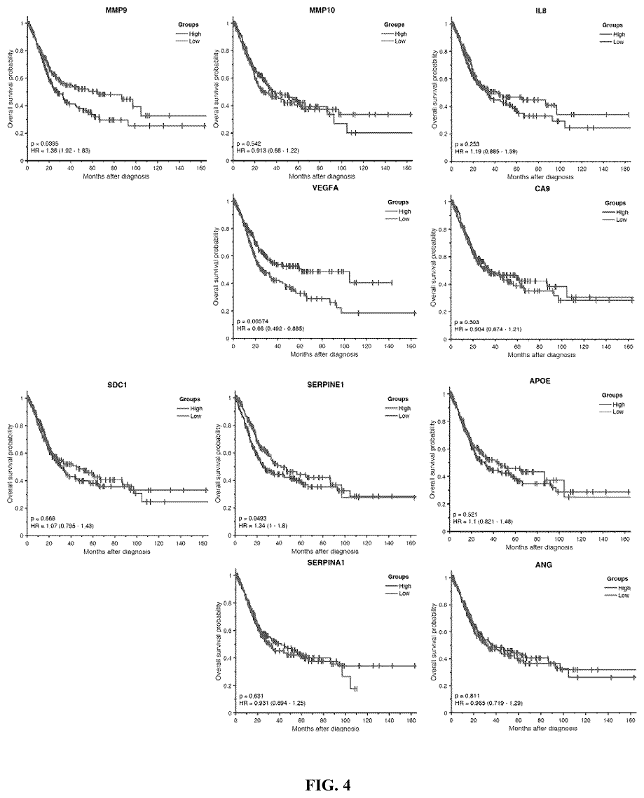

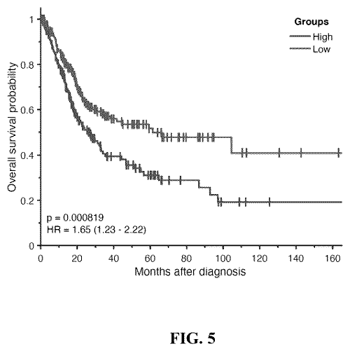

Individually, only low expression of PAI-1 (HR=1.34 (1-1.8), p=0.0493) and MMP9 (HR=1.36 (1.02-1.83), p=0.0395) and high expression of VEGFA (HR=0.66 (0.492-0.885), p=00574) was noted to be associated with improved overall survival probability (FIG. 4).

Collectively, tumors with overall low expression of the 10 analytes compared to high expression were noted to be associated with improved overall survival probability (HR=1.65 (1.23-2.22), p=0.000819) (FIG. 5).

In order to further validate the 10 analytes in bladder cancer, transcript databases from the Black cohort (Seiler et al. Clin Cancer Res. (2019) 25(16): 5082-5093), GSE32894 (Damrauer et al. Proc Natl Acad Sci. (2014) 111(8): 3110-3115) and GSE48075 (Choi et al. Cancer Cell. (2014) 25(2): 152-165) were analyze as described herein.

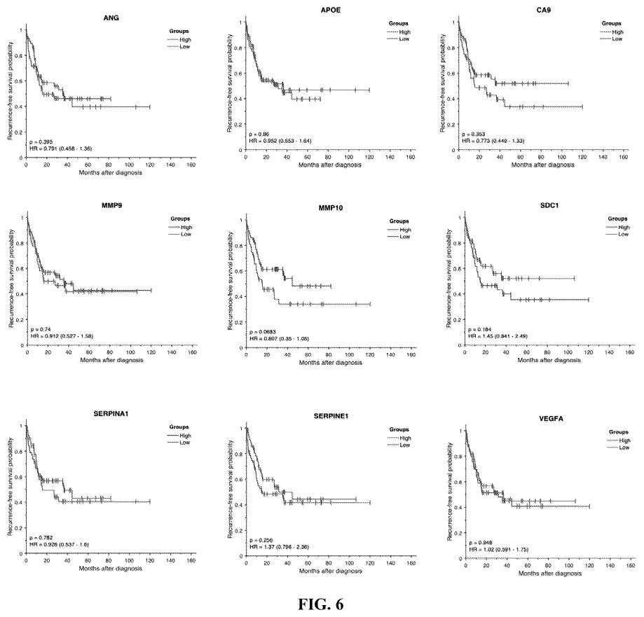

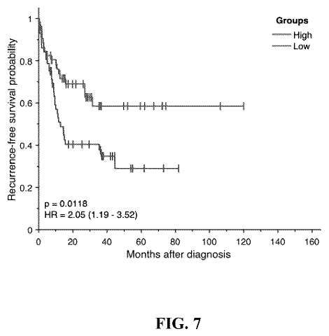

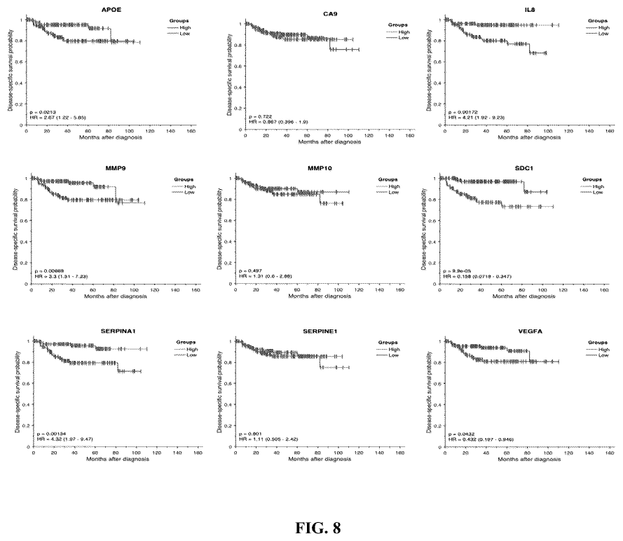

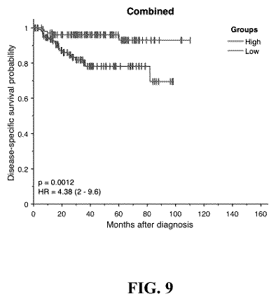

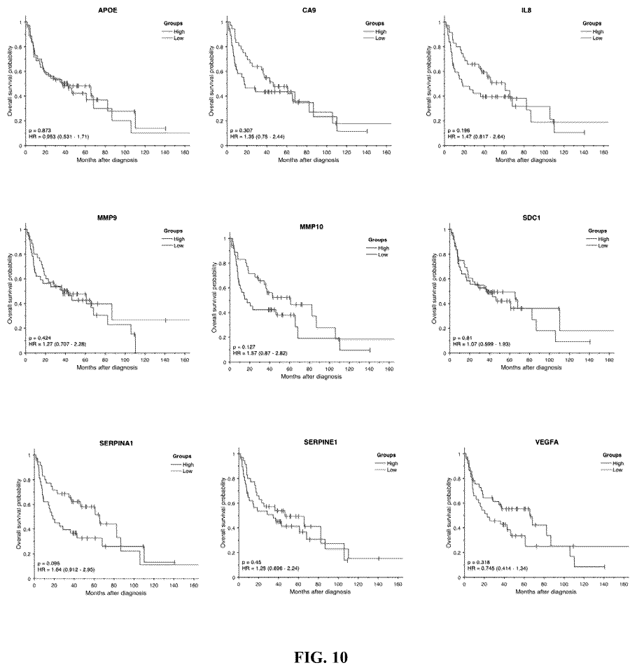

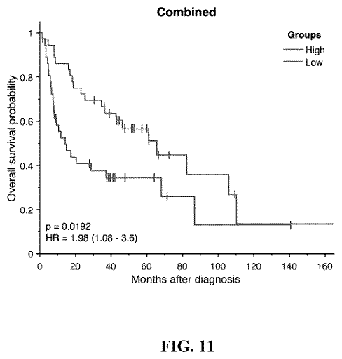

In the Black cohort, only low expression of MMP10 (HR=0.607 (0.35-1.05), p=0.0683) approached significance (FIG. 6) for improved overall survival. Collectively, tumors with overall low expression of the 10 analytes compared to high expression were noted to be associated with improved overall survival probability (HR=2.05 (1.19-3.52), p=0.0118) (FIG. 7). In GSE32894 cohort, only low expression of APOE (HR=2.67 (1.22-5.85), p=0.0213), IL8 (HR=4.21 (1.92-9.23), p=0.00172), MMP9 (HR=3.3 (1.51-7.23), p=0.00669), A1AT (HR=4.32 (1.97-9.47), p=0.00134) and high expression of SDC1 (HR=0.158 (0.0719-0.347), p=0.000099) and VEGFA (HR=0.432 (0.197-0.946), p=0.0432) were associated with improved overall survival (FIG. 8). Collectively, tumors with overall low expression of the 10 analytes compared to high expression were noted to be associated with improved overall survival probability (HR=4.38 (2-9.6), p=0.0012) (FIG. 9). In GSE48075 cohort, only low expression of AAT (HR=1.64 (0.912-2.95), p=0.095) approached significance for improved overall survival (FIG. 10). Collectively, tumors with overall low expression of the 10 analytes compared to high expression were noted to be associated with improved overall survival probability (HR=1.98 (1.06-3.6), p=0.0192) (FIG. 11).

The inventors validated diagnostic molecular signature comprising 10 analytes using an independent, validation sample set of naturally voided urine samples, comprising 37 non-cancer controls and 44 cancer cases (Urquidi et al. Cancer Epidemiol Biomarkers Prev. (2012) 21(12): 2149-58). Target transcripts were measured in urothelial cell RNA samples using quantitative real-time RT-PCR. TaqMan® Low Density Arrays (TLDA) were constructed to include 44 candidate biomarker targets plus 4 selected endogenous controls selected by screening the level of 15 commonly used endogenous controls in the full cohort of samples (described above and below). Biomarker targets were selected primarily from the p-value ranking and molecular signature models described above, but several putative biomarkers were also included (TERT, KRT20, CLU, PLAU, CALR, CA9, ANG). When other selection criteria were equal, genes were selected that encode integral membrane proteins or secreted proteins, because these classes hold potential for development as biomarkers for urinalysis. For quantitative PCR analysis, RNA extraction is performed as described (Urquidi et al. Cancer Epidemiol Biomarkers Prev. (2012) 21(12): 2149-58). Purified RNA samples were evaluated quantitatively and qualitatively using an Agilent Bioanalyzer 2000, prior to storage at −80° C. Complementary DNA was synthesized from 20 to 500 ng of total RNA, depending on availability, using the High Capacity cDNA Reverse Transcriptase Kit (Applied Biosystems, Foster City, CA) following the manufacturer's instructions, with random primers in a total reaction volume of 20 l.

Selection of endogenous reference controls was accomplished by using an aliquot of each sample cDNA in a multiplex PCR preamplification reaction of 15 endogenous reference targets: GAPDH; ACTB; B2M; GUSB; HMBS; HPRT1; IP08; PGK1; POLR2A; PPIA; RPLP0; TBP; TFRC; UBC; YWHAZ. Subsequently, 12.5 μl of the pooled assay mix (0.2×) will be combined with 4 μl of each cDNA sample and 25 μl of the TaqMan® PreAmp Master Mix (2×) in a final volume of 50 μl. Thermal cycling conditions will be as follows: initial hold at 95° C. during 10 min and ten preamplification cycles of 15 sec at 95° C. and 4 min at 60° C. The preamplification products were diluted 1:5 with TE buffer prior to singleplex reaction amplification using the TaqMan® Endogenous Control Array (Applied Biosystems). The reactions will be performed on a 7900HT Fast Real-Time PCR System (AB). Genes with the least variable expression across previous samples (UBC; PPIA; PGK1; GAPDH) were identified using GeNorm software (Integromics, Granada, Spain) and deployed as endogenous controls.

Custom array preamplification and amplification reactions were carried out by constructing TaqMan® Low Density Arrays (TLDA) by Applied Biosystems (AB) using pre-designed assays whose probe would span an exon junction. Targets included were: UBC; PPIA; PGK1; GAPDH (4 endogenous controls); ANG, A1AT, APOE, CA9, IL8, MMP9, MMP10, PAI-1, SDC1 and VEFGA. A multiplex PCR preamplification reaction was performed using the pooled 48 TaqMan® Gene Expression Assays. Assay reagents at 0.2× final concentration were combined with 7.5 μl of each cDNA sample and 15 μl of the TaqMan PreAmp Master Mix (2×) in a final volume of 30 μl. Thermal cycling conditions were as follows: initial hold at 95° C. during 10 min; fourteen preamplification cycles of 15 sec at 95° C. and 4 min at 60° C. and a final hold at 99.9° C. for 10 min. Ten microliters of undiluted preamplification products was used in the subsequent singleplex amplification reactions, combined with 50 μl of 2× TaqMan® Universal PCR MasterMix (AB) in a final volume of 100 μl, following manufacturer's instructions. One sample of Human Universal Reference Total cDNA (Clontech) was included as a calibrator in each micro-fluidic card. The reactions were run in a 7900HT Fast Real-Time PCR System (AB).

Real-time PCR amplification results were processed with RQ manager (AB) and StatMiner (Integromics) software packages. The baseline correction was manually checked for each target, and the Ct threshold was set to 0.2 for every target across all plates. Delta-Delta CT values were calculated using a geometric average of the four endogenous reference targets (UBC, PPIA, PGK1, and GAPDH) as normalizer and Human Universal Reference Total cDNA (Clontech) as the calibrator. Genes deemed to be differentially expressed were determined by t-test comparison (p<0.01).

Derivation of optimal diagnostic molecular signatures was accomplished by using L1-regularized logistic regression to establish a prediction model, i.e., to predict the actual status of a given sample as cancer or control. Details of the strategy, optimal solution parameters, and application of a fast implementation approach for L1 regularized learning algorithms will be presented in the following. Due to the small sample size, the leave-one-out cross validation (LOOCV) method was adopted to estimate the prediction performance. Lodewyk et al. Bioinformatics (2005) 21(19): 3755-3762. In each iteration, one sample was held out for test, and the remaining samples were used for training. The regularization parameter k was first estimated through ten-fold cross validation using the training data, and then a predictive model was trained using the estimated parameter and blindly applied to the held-out sample.

The experiment was repeated until all samples had been tested. An ROC curve was then plotted to visualize how a prediction model performed at different sensitivity and specificity levels, and the area under receiver operating characteristic curves (AUC) was reported. To verify the data, a permutation test was also performed to estimate the p value of predictive performance. The permutation test was repeated 1000 times. In each iteration, the class labels were randomly shuffled, the above-described experimental protocol was executed, and the area under the resulting ROC curve was recorded. The p value was computed as the occurrence frequencies of the iterations where the resulting AUCs outperformed that obtained using the original class labels. Ap value <0.01 was considered to be statistically significant. Statistical analyses were performed by SPSS 13.0, and by MedCalc version 8.0 (MedCalc Software, Mariakerke, Belgium).

Differential expression values were calculated by normalization using the reference targets (UBC, PPIA, PGK1, and GAPDH) and Human Universal Reference Total cDNA (Clontech) as the calibrator on each plate. KM curves were plotted to visualize how each prediction model performed with low vs. high expression of the 10 analytes.

### Example 2

A Diagnostic Gene Expression Signature for Bladder Cancer can Stratify Cases into Prescribed Molecular Subtypes and Predict Outcome

### Materials and Methods

### Data Acquisition

A discovery cohort comprised of 430 samples from TCGA with gene transcriptome data of which 404 patients had valid survival data (19 normal and 411 cancer). The dataset includes only one non-muscle invasive bladder cancer (NMIBC) with the rest being muscle invasive bladder cancer (MIBC) patients. Three additional datasets were accessed for validation analyses: GSE87304; including 303 MIBC patients with the primary outcome of recurrence free survival (Seiler et al. Eur Urol. (2017) 72: 544-554), GSE48075; including 142 NMIBC patients

TABLE 4
Demographic and clinical-pathologic characteristics of study cohorts

| | | TCGA | | GSE87304 | | GSE48075 | | GSE32894 | |
|---|---|---|---|---|---|---|---|---|---|
| | | (N = 412) | | (N = 323) | | (N = 73) | | (N = 308) | |
| Variable | Value | n | % | n | % | n | % | n | % |
| Age | <65 | 151 | 37.0 | 182.0 | 56.0 | 22.0 | 30.0 | 100 | 32.0 |
| | ≥65 | 261 | 63.0 | 136.0 | 42.0 | 51.0 | 70.0 | 208 | 68.0 |
| Sex | Female | 304 | 74.0 | 235.0 | 73.0 | 54.0 | 74.0 | 228 | 74.0 |
| | Male | 108 | 26.0 | 88.0 | 27.0 | 19.0 | 26.0 | 80 | 26.0 |
| Race | White | 327 | 79.0 | — | — | 54.0 | 74.0 | — | — |
| | Other | 85 | 21.0 | — | — | 19.0 | 26.0 | — | — |
| Stage | <=I | 3 | 0.0 | 0.0 | 0.0 | 0.0 | 0.0 | 213 | 69.0 |
| | II | 121 | 29.0 | 148.0 | 46.0 | 37.0 | 51.0 | 85 | 28.0 |
| | III | 196 | 48.0 | 123.0 | 38.0 | 16.0 | 22.0 | 7 | 2.0 |
| | IV | 59 | 14.0 | 0.0 | 0.0 | 6.0 | 8.0 | 1 | 0.0 |
| Grade | Low | 21 | 94.0 | — | — | — | — | 153 | 50.0 |
| | High | 388 | 5.0 | — | — | — | — | 155 | 50.0 |

with the primary outcome of overall survival (Choi et al. Cancer Cell (2014) 25: 152-165), and GSE32894; including 215 NMIBC and 93 MIBC patients (Damrauer et al. Proc Natl Acad Sci USA (2014) 111: 3110-3115) patients with the primary outcome of disease specific survival, respectively. These datasets are an open resource with no noted ethical issues. The study populations within these four cohorts are presented in Table 4. Briefly, TCGA largely had MIBC treated by cystectomy, GSE87304 had MIBC treated with neoadjuvant chemotherapy (NAC) prior to cystectomy, GSE48075 had a mix of NMIBC and MIBC treated with or without NAC and GSE32894 had transurethral resection of bladder tumor (TURBT).

### Data Processing and Analysis

Bladder urothelial carcinoma Illumina Hi-Seq counts from TCGA were downloaded from the Genomic Data Commons (GDC) data portal, and corresponding clinical annotation including survival information was accessed via the TCGA Clinical Data Resource. Consensus MIBC classifications of TCGA cases were obtained from the consensus MIBC study. A comprehensive analysis using the edgeR package was performed to obtain the gene expression values (Robinson et al. Bioinformatics (2010) 26: 139-140.

### Survival Analysis

Kaplan-Meier curves were used to determine the association between individual biomarkers (low vs. high expression) and prognosis. High expression was defined as >median, and low expression was defined as <median.

### Univariate and Multivariate Analysis

The biomarkers associated with each multiplex test were evaluated by univariate Cox regression, and the relevant biomarkers were then evaluated using a multivariate Cox regression model to select the biomarkers that were most strongly associated with survival. All statistical analyses were performed using SPSS19.0.

### KEGG Pathway Analysis

The Database for Annotation, Visualization, and Integrated Discovery (DAVID, david.ncifcrf.gov/) was used to perform Gene Ontology (GO) functional analyses (Dennis et al. Genome Biol. (2003) 4: P3), reporting the top biological processes and cellular components.

### Results

Using a series of gene expression datasets from TCGA and the GEO, the inventors evaluated the association of the 10 biomarkers (ANG, APOE, A1AT, CA9, IL8, MMP9, MMP10, PAI1, SDC1 and VEGFA) comprising the diagnostic signature with prescribed bladder cancer molecular subtypes described herein and with clinical outcomes. The TCGA cohort was parsed into luminal (n=242, 59%) and basal (n=166, 41%) subtypes based on gene expression profiles (FIG. 3A) (Choi et al. Cancer Cell (2014) 25: 152-165). Analyses showed that 79% of samples in the luminal subtype showed high expression of VEGFA (p=4.36e-8) and SDC1 (1.62-13) (FIG. 3B). Notably, tumors with a papillary morphology were significantly enriched in the luminal subtype (luminal 80% vs. basal 20%; p=1.29e-4). Conversely, 83% of samples in the basal subtypes had high expression of MMP9 (p=1.49e-29), MMP10 (p=1.14e-2), IL8 (p=1.52e-8), SERPINE1 (p=2.7e-9), APOE (p=1.05e-10) and SERPINA1 (p=2.04e-23) (FIG. 3B). The basal subtype was enriched with tumors of higher stage (T2-4, 93% vs. Ta and T1, 7.3%; p=5.4e-34).

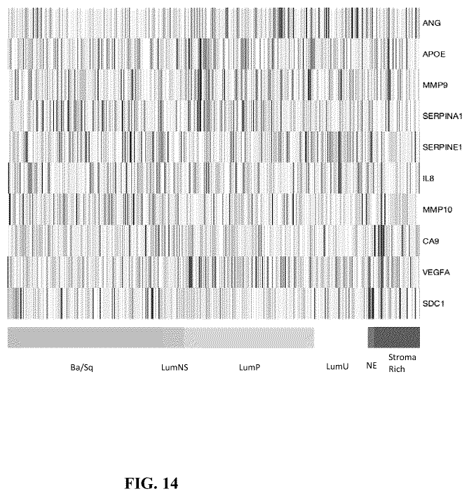

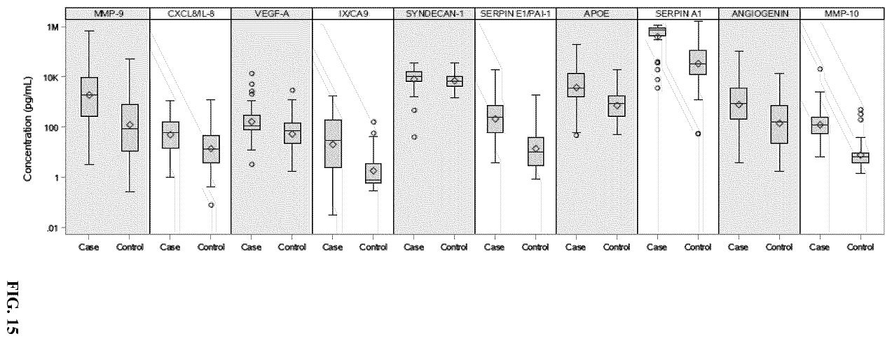

The inventors also tested whether the subset of biomarkers described herein were differentially expressed with respect to a more contemporary consensus set (Kamoun et al. Eur Urol (2020) 77: 420-433) of six molecular classes of bladder cancer: luminal papillary, luminal non-specified, luminal unstable, stroma-rich, basal/squamous, and neuroendocrine-like. Though there were limited subjects in some of the molecular classes (e.g., neuroendocrine-like and luminal non-specified), analyses showed that the subset of biomarkers described herein could segregate samples into the six consensus subtypes (FIG. 14). Together, these findings show that the expression patterns of the subset of biomarkers described herein are associated with reported molecular subtypes of bladder cancer.

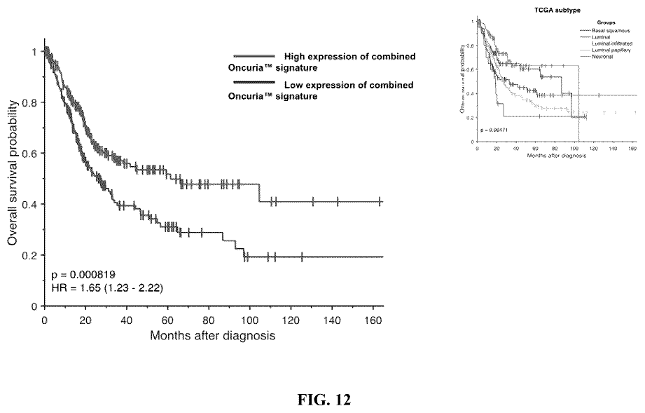

Reported molecular subtypes have been reported to be associated with overall survival. Here, Kaplan-Meier analysis of the TCGA subjects indicated that high expression of the subset biomarker signature described herein (ANG, APOE, A1AT, CA9, IL8, MMP9, MMP10, PAI1, SDC1 and VEGFA) was correlated with a significant reduction in overall survival (FIG. 12; HR=1.65; p=0.000819). Analysis of each of the 10 individual biomarkers described herein (ANG, APOE, A1AT, CA9, IL8, MMP9, MMP10, PAI1, SDC1 and VEGFA) revealed that high expression of MMP9 (HR=1.36; p=0.0395), and SERPINE1 (HR=1.34; p=0.0493) were associated with a significant reduction in overall survival, while high expression levels of VEGFA (HR=0.66; p=0.00574) were associated with a significant improvement in overall survival (FIG. 4). Table 5 reports the Cox univariate and multivariate analysis, with VEGFA, MMP9, SERPINA1 and SERPINE1 being associated with survival probabilities.

TABLE 5
Coefficients based on a Cox regression analysis
of the 10 Oncuria ™biomarkers

| | Univariate analysis | | | Multivariate analysis | | |
|---|---|---|---|---|---|---|
| | | 95% CI | p- | | 95% CI | p- |
| Variables | HR | of HR | value | HR | of HR | value |
| SERPINA1 | 0.96 | [0.82, 1.12] | 0.57 | 0.76 | [0.60, 0.95] | 0.02 |
| ANG | 1.00 | [0.87, 1.16] | 0.97 | 1.01 | [0.86, 1.17] | 0.94 |
| APOE | 1.08 | [0.93, 1.25] | 0.34 | 0.94 | [0.75, 1.17] | 0.57 |
| CA9 | 0.94 | [0.82, 1.08] | 0.39 | 1.03 | [0.87, 1.21] | 0.76 |
| IL8 | 1.06 | [0.91, 1.23] | 0.46 | 1.06 | [0.88, 1.27] | 0.57 |
| MMP9 | 1.21 | [1.04, 1.41] | 0.02 | 1.31 | [1.04, 1.65] | 0.02 |
| MMP10 | 0.95 | [0.82, 1.11] | 0.54 | 0.92 | [0.78, 1.08] | 0.31 |
| SERPINE1 | 1.15 | [0.99, 1.33] | 0.08 | 1.17 | [0.98, 1.39] | 0.08 |
| SDC1 | 1.00 | [0.86, 1.16] | 0.95 | 1.01 | [0.86, 1.19] | 0.88 |
| VEGFA | 0.81 | [0.69, 0.94] | 0.01 | 0.79 | [0.66, 0.94] | 0.01 |

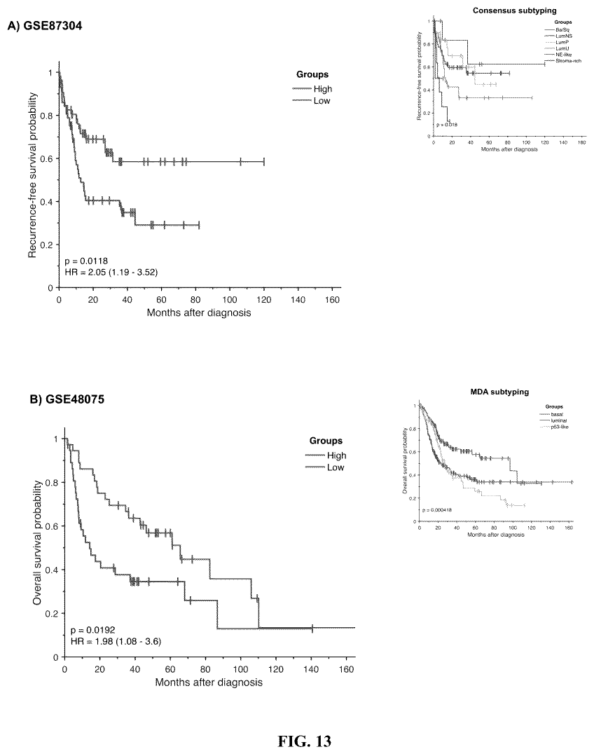

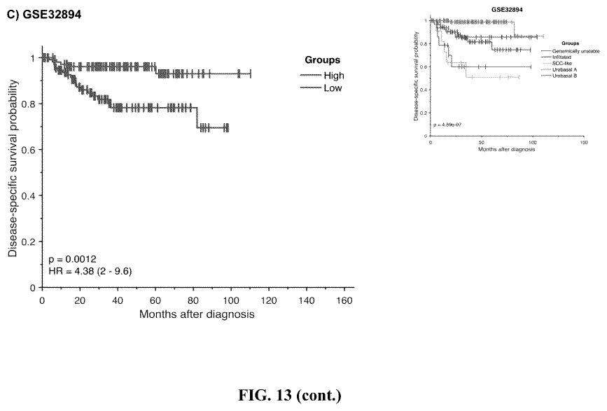

Validation studies were performed using three independent, publicly available datasets (GSE87304, GSE48075, GSE32894). Nine of the 10 biomarkers described herein (ANG, APOE, A1AT, CA9, IL8, MMP9, MMP10, PAI1, SDC1 and VEGFA) were present in each dataset. Notably, IL-8 was not present in GSE87304 and ANG was not present in GSE48075 and GSE32894. Similar to the analysis of TCGA data, we found that tumors with a relatively low expression of the combined biomarker signature described herein (ANG, APOE, A1AT, CA9, IL8, MMP9, MMP10, PAI1, SDC1 and VEGFA) were associated with improved survival probabilities in all three datasets (GSE87304, recurrence-free survival probability, HR=2.05 (1.19-3.52), p=0.0118; GSE48075, overall-survival probability, HR=1.98 (1.06-3.6), p=0.0192; GSE32894, disease-specific survival probability, HR=4.38 (2-9.6), p=0.0012) (FIG. 13A-C, respectively). In the GSE87304 cohort, only the low expression of MMP10 (HR=0.607 (0.35-1.05), p=0.0683) approached significance for improved recurrence-free survival (Supplemental FIG. 2A). In the GSE48075 cohort, only the low expression of SERPINA1 (HR=1.64 (0.912-2.95), p=0.095) approached significance for improved overall survival (Supplemental FIG. 2B). In the GSE32894 cohort, the low expression of APOE (HR=2.67 (1.22-5.85), p=0.0213), IL8 (HR=4.21 (1.92-9.23), p=0.00172), MMP9 (HR=3.3 (1.51-7.23), p=0.00669), SERPINA1 (HR=4.32 (1.97-9.47), p=0.00134), and the high expression of SDC1 (HR=0.158 (0.0719-0.347), p=0.000099) and VEGFA (HR=0.432 (0.197-0.946), p=0.0432) were associated with improved disease-specific survival (Supplemental FIG. 2C). Taken together, these findings validate the notion that multiplex signatures provide better prognostic models than individual biomarkers. Prospective validation in a larger cohort may lead to the derivation of a weighted algorithm that would maximize the utility of molecular signature(s) for subtyping and prognosis. Subsequently, GO enrichment analysis indicated that the biomarkers associated with the bladder cancer diagnostic signature were significantly enriched in the regulation of induction of positive chemotaxis and vascular permeability affecting the extracellular matrix, key processes in the growth of tumors.

TABLE 6
Top biological processes and cellular component reported in the gene ontology pathway analysis
of differentially expressed genes associated with a bladder cancer associated signature

| GO-term | Description | Count in network | Strength | False discovery rate |
|---|---|---|---|---|
| | Biological Process (Gene Ontology) | | | |
| GO: 0050930 | Induction of positive | 2 of 15 | 2.42 | 0.0301 |
| | chemotaxis | | | |
| GO: 0043117 | Positive regulation of vascular | 2 of 16 | 2.39 | 0.0304 |
| | permeability | | | |
| GO: 0043116 | Negative regulation of vascular | 2 of 18 | 2.34 | 0.0304 |
| | permeability | | | |
| GO: 0030225 | Macrophage differentiation | 2 of 26 | 2.18 | 0.0385 |
| GO: 0002690 | Positive regulation of | 3 of 98 | 1.78 | 0.0210 |
| | leukocyte chemotaxis | | | |
| | Cellular Component (Gene Ontology) | | | |
| GO: 0005615 | Extracellular space | 9 of 3195 | 0.74 | 0.00087 |

### Discussion

Several attempts have been made to identify panels of biomarkers for potential cancer detection, but these studies have relatively small sample size, limited populations (i.e., few benign, confounding conditions included) and have not undergone extensive validation. This belies the inherent difficulty in identifying, characterizing, and validating a subset of biomarkers for bladder cancer that have high sensitivity and accuracy.

Urothelial carcinoma is pathologically classified as non-muscle-invasive bladder cancer (NMIBC) or muscle-invasive bladder cancer (MIBC). The standard treatment for NMIBC is transurethral resection of bladder tumor (TURBT) for low-risk cases, or TURBT followed by intravesical therapy, such as BCG, for high-risk NMIBC, and the universal treatment for MIBC is radical cystectomy. A considerable number of NMIBC patients (50% to 80%) have tumor recurrence (van der Heijden & Witjes European Urology Supplements (2009) 8: 556-562) and up to 45% progress to MTBC after 5 years, leading to poor survival rates associated with more advanced disease. Pathological staging is a key factor in current clinical decision making and prognosis of bladder cancer; nevertheless, the clinical outcomes of patients with the same stage often differ, indicating that the current staging system is not sufficient to reflect biological heterogeneity, and accurately determining the prognosis of patients is challenging. Prognostic evaluation models based on molecular signatures or subtypes may be able to better guide individualized treatment and improve outcome prediction.

The inventors analyses showed that the levels of biomarkers within a diagnostic test using the subset of biomarkers described herein could stratify patients into luminal (VEGFA (p=4.36e-8) and SDC1 (p=1.62e-13) or basal (MMP9 (p=1.49e-29), MMP10 (p=1.14e-2), IL8 (p=1.52e-8), SERPINE1 (p=2.7e-9), APOE (p=1.05e-10) and SERPINA1 (p=2.04e-23) subtypes. Furthermore, survival curve analysis showed that multivariate models comprised of specific biomarkers described herein (VEGFA, MMP9, SERPINA1 and SERPINE1) were associated with outcome. Validation analyses using three publicly available datasets (GSE87304, GSE48075, GSE32894) confirmed that multiplex signatures provided better prognostic models than individual biomarkers (FIGS. 4, 12, 13). Lastly, the GO enrichment analysis indicates enriched biomarker activity within the extracellular space (Table 6). The immunostaining patterns of the subset of biomarkers described herein are enriched in human bladder stromal tissues in malignancy and associated with a reduction in overall survival.

In summary, the inventors demonstrated that the biomarkers comprise an established diagnostic signature have value for molecular subtyping and prediction of clinical outcomes for patients with bladder cancer. Specifically, patients with high expression of the biomarker signature described herein were associated with a significant reduction in overall survival.

### Example 3

### Diagnostic Accuracy of Biomarker Panel for Detection of Upper Tract Urothelial Tumors

### Summary

To evaluate the performance of a multiplex immunoassay capable of querying a voided urine sample for 10 protein biomarkers associated with urothelial carcinoma A1AT, APOE, ANG, CA9, IL8, MMP9, MMP10, PAI1, SDC1 and VEGFA. Its application to UTUC was evaluated in a multi-institutional cohort of 31 prospectively collected subjects presenting for evaluation of upper tract mass along with 41 prospective collected matched controls (i.e., non-tumor bearing). The ability of the test to identify patients harboring UTUC was assessed. UTCU status was confirmed by endoscopy and tissue biopsy or definitive surgery. Diagnostic performance was assessed using ROC curves.

The multiplex immunoassay described herein consisting of A1AT, APOE, ANG, CA9, IL8, MMP9, MMP10, PAI1, SDC1 and VEGFA showed an AUC of 0.897 (95% CI: 0.817-0.977) with an overall sensitivity of 93.5%, specificity of 75.6%, NPV 93.9% and PPV 74.4%. Sensitivity values of the diagnostic panel for high-grade UTUC, low-grade UTUC, non-invasive UTUC and invasive UTUC were 88.9%, 92.3%, 86.7% and 100%, respectively. Urinary cytology or selective ureteral washing/cytology was associated with an overall sensitivity of 58.3%, specificity of 100%, NPV 79.2% and PPV 100%. Sensitivity values of cytology for high-grade UTUC, low-grade UTUC, non-invasive UTUC and invasive UTUC were 50%, 100%, 80% and 42.9%, respectively.

Urinary levels of the biomarker panel consisting of A1AT, APOE, ANG, CA9, IL8, MMP9, MMP10, PAI1, SDC1 and VEGFA provided for the accurate discrimination of UTUC and controls non-tumor bearing individuals. The multiplex immunoassay test described herein can achieve the efficient and accurate detection of UTUC in a non-invasive patient setting.

Despite advances in technology, diagnosis of upper tract tumors continues to be challenging and often cytologies and/or biopsies are inconclusive or not performed due to the difficulty of reaching the lesion of concern. Consequently, the development of an accurate diagnostic assay that could be applied to non-invasively obtained urine samples would benefit both patients and health care systems. In this study, we tested the potential clinical utility of the multiplex immunoassay described herein for the detection of UTUC bladder in a prospectively recruited cohort of patients who presented for urological evaluation at three institutions. The multiplex immunoassay described herein achieved a strong overall diagnostic performance, achieving an AUC of 0.897 (95% CI: 0.817-0.977) with an overall sensitivity and specificity values of 93.5% and 75.6%, respectively, and a negative predictive value (NPV) and positive predictive value (PPV) of 93.9% and 74.4%, respectively. The multiplex immunoassay described herein shows promise for clinical application in the non-invasive evaluation of patients suspected of harboring UTUC.

### Methods

### Patient Characteristics.

Under Institutional Review Board approval and informed consent, voided urine samples, and associated coded clinical information were collected in a tissue bank at Cedars-Sinai Medical Center (Los Angeles, CA) and Kindai University (Osaka, Japan). The tissue banks were queried for subjects with biopsy proven UTUC (n=31) and matched controls (n=41). The control cohort consisted of 41 subjects with no previous history of UTUC (microscopic hematuria, n=7; gross hematuria, n=4; urinary tract infection, n=1; voiding symptoms, n=6, history of bladder cancer, n=4, prostate cancer, n=1, kidney stones, n=5; control, n=13) who were matched for age, gender, and race. In our cancer subjects as well as control subjects with hematuria, axial imaging of the abdomen and pelvis with and without intravenous contrast was performed in addition to cystoscopy. In subjects with an abnormality noted on upper tract imaging or an abnormality on cystoscopy, a formal evaluation was performed in the operating room under anesthesia. This evaluation consisted of cystoscopy for bladder only lesions or cystoscopy and ureteroscopy for upper tract lesions. All the cancer subjects had documented urothelial cell carcinoma confirmed by histological examination of excised tumor tissue (biopsy and/or nephroureterectomy). Pertinent information on clinical presentation, staging, histologic grading, and outcome were recorded (Table 7).

TABLE 7
Patient Characteristics

| | | TOTAL | | CASES | | CONTROLS | | |
|---|---|---|---|---|---|---|---|---|
| | | (N = 72) | | (N = 31) | | (N = 41) | | |
| Variable | | n | % | n | % | n | % | P |
| Age | 18-54 | 6 | 8.3 | 2 | 6.5 | 4 | 9.8 | .97 |
| | 55-64 | 3 | 4.2 | 1 | 3.2 | 2 | 4.9 | |
| | 65-74 | 31 | 43.1 | 14 | 45.2 | 17 | 41.5 | |
| | 75+ | 32 | 44.4 | 14 | 45.2 | 18 | 43.9 | |
| Sex | Female | 23 | 31.9 | 10 | 32.3 | 13 | 31.7 | .99 |
| | Male | 49 | 68.1 | 21 | 67.7 | 28 | 68.3 | |
| Race | Asian | 57 | 79.2 | 26 | 83.9 | 31 | 75.6 | .56 |
| | White | 15 | 20.8 | 5 | 16.1 | 10 | 24.4 | |
| Cytology | Negative | 24 | 33.3 | 5 | 16.1 | 19 | 46.3 | .0005 |
| | Positive | 7 | 9.7 | 7 | 22.6 | 0 | 0.0 | |
| | unknown | 41 | 56.9 | 19 | 61.3 | 22 | 53.7 | |
| Location | Pelvis | 11 | 15.3 | 11 | 35.5 | 0 | 0.0 | |
| | Ureter | 18 | 25.0 | 18 | 58.1 | 0 | 0.0 | |
| | unknown | 2 | 2.8 | 2 | 6.5 | 0 | 0.0 | |
| | N/A | 41 | 56.9 | 0 | 0.0 | 41 | 100.0 | |
| Stage | T0 | 7 | 9.7 | 7 | 22.6 | 0 | 0.0 | |
| | T1 | 8 | 11.1 | 8 | 25.8 | 0 | 0.0 | |
| | T2-3 | 16 | 22.2 | 16 | 51.6 | 0 | 0.0 | |
| | N/A | 41 | 56.9 | 0 | 0.0 | 41 | 100.0 | |
| Grade | Low | 13 | 18.1 | 13 | 41.9 | 0 | 0.0 | |
| | High | 9 | 12.5 | 9 | 29.0 | 0 | 0.0 | |
| | unknown | 9 | 12.5 | 9 | 29.0 | 0 | 0.0 | |
| | N/A | 41 | 56.9 | 0 | 0.0 | 41 | 100.0 | |

### Specimen Collection and Processing.

Prior to any type of therapeutic intervention, 50-100 mL of voided urine was obtained from each subject. Fifty milliliters of urine was used for clinical laboratory analyses (e.g., urinary cytology and urinalysis) per standard procedures. The remaining urine aliquot was assigned a unique identifying number before immediate laboratory processing. Each urine sample was centrifuged at 600×g 4° C. for 5 min. The supernatant was decanted and aliquoted, while the urinary pellet was snap frozen. Both the supernatant and pellet were stored at −80° C. prior to analysis. Aliquots of urine supernatants were thawed and analyzed for protein content using a Pierce 660-nm Protein Assay Kit (Thermo Fisher Scientific Inc., Waltham, MA, USA).

### Multiplex Testing.

The concentrations of the 10 proteins (A1AT, APOE, ANG, CA9, IL8, MMP9, MMP10, PAI1, SDC1 and VEGFA) were monitored using an analytically validated multiplex bead-based immunoassay described herein (Oncuria®) from R&D Systems Inc. (Minneapolis, MN) for Luminex 200. Urine samples were passively thawed, centrifuged for 10 minutes×1,000 g. Urine samples were passively thawed and handled on ice prior to diluting 2-fold with R&D Assay Diluent. Samples, standards, and controls (50 l) were added to the 96 well plate in duplicate. The multiplex immunoassay was conducted according to the manufacturer's instructions. A seven-point standard curve across the 4 log dynamic range of the assays was included in the current assay design. Plates were read on the Luminex® 100/200 (Luminex Corp, Austin, TX). Calibration curves were generated along with optimal fit in conjunction with Akaike's information criteria (AIC) values.

### Statistical Analysis.

Fisher's exact tests determined associations between key demographic features (age, sex, race, cytology) and cancer status. We applied the previous 10-biomarker molecular signature, and the previous molecular signature with the three demographic variables (age, sex, and race), to these new data and assessed the fit. Statistical significance in this study was set at p<0.05 and all reported p values were 2-sided. All analyses were performed using SAS software version 9.4 (SAS Institute Inc., Cary, NC).

### Results

In line with the demographics of the participating institutions, many of the study subjects were elderly, Asian males. Cytology was available in 12 of the 31 cancer cohort and 19 of the 41 controls with a reported sensitivity of 58.3% and specificity of 100%. No subjects in the control cohort had an abnormal axial imaging. Furthermore, in follow-up, none of the control subjects were noted to develop urothelial carcinoma or a gross hematuria event. In the cancer cohort, 48.4% of subjects had non-muscle invasive disease and 41.9% of subjects had low-grade disease. Demographic, clinical, and pathologic characteristics of the cohort are presented in Table 7.

The concentration of the ten proteins measured in urine is presented in Table 8.

TABLE 8
Mean urinary (±SD) concentrations of 10 biomarkers assessed by
the multiplex immunoassay described herein in cohort of 72 subjects

| | | CASES | | | | | |
|---|---|---|---|---|---|---|---|
| Biomarker | Detectable | (N = 31) | | | | | |
| pg/mL | % | n | mean | sd | min | median | max |
| MMP-9 | 88.9 | 30 | 50,945 | 151,498 | 3.24 | 2,056 | 653,889 |
| CXCL8/IL-8 | 94.4 | 29 | 173.3 | 293.8 | 1.75 | 60.3 | 1,122 |
| VEGF-A | 95.8 | 30 | 484.1 | 1,083 | 3.30 | 112.7 | 5,272 |
| IX/CA9 | 77.8 | 27 | 188.8 | 409.5 | 0.64 | 33.8 | 2,029 |
| Syndecan-1 | 100.0 | 31 | 12,187 | 8,543 | 40.6 | 10,615 | 36,600 |
| PAI-1 | 90.3 | 30 | 1,428 | 3,661 | 6.14 | 268.0 | 18,997 |
| ApoE | 97.2 | 31 | 17,692 | 41,011 | 47.6 | 3,562 | 200,704 |
| A1AT | 91.7 | 31 | 659,816 | 351,121 | 3,683 | 761,076 | 1,175,337 |
| Angiogenin | 93.1 | 30 | 1,765 | 2,038 | 3.93 | 839.2 | 7,078 |
| MMP-10 | 83.3 | 28 | 1,025 | 3,893 | 7.52 | 122.9 | 20,710 |

| | CONTROLS | | | | | | |
|---|---|---|---|---|---|---|---|
| Biomarker | (N = 41) | | | | | | |
| pg/mL | n | mean | sd | min | median | max | P |
| MMP-9 | 34 | 5,705 | 14,030 | 0.26 | 165.0 | 53,143 | .11 |
| CXCL8/IL-8 | 39 | 110.4 | 244.7 | 0.07 | 13.7 | 1,225 | .35 |
| VEGF-A | 39 | 218.8 | 513.8 | 2.47 | 71.0 | 2,967 | .22 |
| IX/CA9 | 29 | 14.5 | 32.2 | 0.03 | 1.64 | 161.3 | .04 |
| Syndecan-1 | 41 | 8,810 | 6,521 | 1,522 | 7,140 | 36,032 | .07 |
| PAI-1 | 35 | 113.8 | 331.3 | 0.74 | 17.0 | 1,870 | .06 |
| ApoE | 39 | 1,939 | 3,486 | 65.1 | 904.0 | 19,285 | .04 |
| A1AT | 35 | 99,455 | 174,979 | 1,214 | 32,960 | 701,868 | .0000 |
| Angiogenin | 37 | 1,047 | 2,379 | 0.48 | 238.0 | 13,731 | .19 |
| MMP-10 | 32 | 39.3 | 104.3 | 0.60 | 6.33 | 486.9 | .19 |

Median urinary levels of MMP9 (50,945 vs. 5,705), IL8 (173.3 vs. 110.4), VEGF-A (484.1 vs. 218.8), CA9 (188.8 vs. 14.5), SDC1 (12,187 vs. 8810), PAI1 (1,428 vs. 113.8), ApoE (17,692 vs. 1,939) A1AT (659,816 vs. 99,455), ANG (1,765 vs. 1,047) and MMP10 (1,025 vs 39.3) were higher in subjects with UTUC compared to controls with significance being reached for CA9, ApoE and A1AT. A box plot illustrating the levels of the 10 biomarker levels in the cases and control is presented in FIG. 16.

The ability of the 10 biomarkers (A1AT, APOE, ANG, CA9, IL8, MMP9, MMP10, PAI1, SDC1 and VEGFA) to predict the presence of UTUC was analyzed using Youden Index cutoff value and nonparametric ROC analysis. The 10 biomarker signature achieved an AUC of 0.897 (95% CI. 0.817-0.977) with an overall sensitivity of 93.5%, specificity of 75.6%, NPV of 93.9%, PPV of 74.4% and accuracy of 83.3%.

Table 9 denotes the overall sensitivity and specificity achieved using the Oncuria® hybrid signature for low grade and high grade, and non-muscle invasive bladder cancers and muscle invasive bladder cancers.

TABLE 9
Summary of diagnostic performance of the multiplex immunoassay described herein
vs. cytology in high-grade/low-grade and high stage/low stage bladder cancer

| | | | TRUE | | FALSE | | | | | | | | 95% | 95% |
|---|---|---|---|---|---|---|---|---|---|---|---|---|---|---|
| Model | | N | Positive | Negative | Positive | Negative | Sensitivity | Specificity | NPV | PPV | Accuracy | AUC | LCL | UCL |
| Oncuria ® | Overall | 72 | 29 | 31 | 10 | 2 | 93.5 | 75.6 | 93.9 | 74.4 | 83.3 | 89.7 | 81.7 | 97.7 |
| | Low Grade | 13 | 12 | | | 1 | 92.3 | | | | | | | |
| | High Grade | 9 | 8 | | | 1 | 88.9 | | | | | | | |
| | Stage 0-1 | 15 | 14 | | | 1 | 93.3 | | | | | | | |
| | Stage 2-3 | 16 | 15 | | | 1 | 93.8 | | | | | | | |
| Cytology | Overall | 31 | 7 | 19 | 0 | 5 | 58.3 | 100.0 | 79.2 | 100.0 | 83.9 | 79.2 | 64.6 | 93.7 |
| | Low Grade | 2 | 2 | | | 0 | 100.0 | | | | | | | |
| | High Grade | 4 | 2 | | | 2 | 50.0 | | | | | | | |
| | Stage 0-1 | 5 | 4 | | | 1 | 80.0 | | | | | | | |
| | Stage 2-3 | 7 | 3 | | | 4 | 42.9 | | | | | | | |

### Discussion

Patients suspected of harboring UTUCs are usually evaluated with computed tomography (CT), retrograde pyelography (RGP) and upper tract urinary cytology. Imaging studies usually reveal a filling defect or an obstructive mass, which is often associated with hydronephrosis, hydroureter or renal stones. Despite being non-invasive methods, imaging studies, which tend to have a low sensitivity, are not used as the sole diagnostic test for UTUC, since numerous causes exist to explain a filling defect other than UTUC (Chlapoutakis et al. Eur J Radiol. (2010) 73: 334-8). Urovysion (Sassa et al. Am J Clin. Pathol. (2019) 151(5): 469-478), NMP22 (Yafi et al. Urologic Oncology: Seminars and Original Investigations. 2015; 33(2):66.e25-66.e31) and ImmunoCyt (Lodde et al. Urology 58: 362-366) all are reported to have either low sensitivity, low specificity, or both for detecting UTUC, therefore cystoscopy with ureteroscopy and renal washings or biopsy, all invasive procedures, are standard of care in the evaluation of patients with UTUC. Due to the above-mentioned shortcomings, the identification of sensitive non-invasive molecular markers for the detection of UTUC is urgently required.

The multiplex assay described herein has advantages including reduced cost through lower labor needs and reagent consumption, and the generation of more data with less sample, but the major advantage is the potential to significantly improve clinical test sensitivity and specificity by a combination of multiple biomarkers. Recently, several groups have begun to identify panels of diagnostic biomarkers for potential bladder cancer application; Hoque et al. reported on the methylation of four genes (CDKN2A, ARF, MGMT, GSTP1) (J Natl. Cancer Inst (2006) 98(14): 996-1004), Chung et al. reported on the methylation of selected five target genes (MYO3A, CA10, NKX6-2, DBC1, SOX11) (Cancer Epidemiol Biomarkers Prev. (2011) 20(7): 1483-91), Hanke et al. reported on two mRNA genes (ETS2, uPA) (Clin Chem. (2007) 53(12): 2070-7), Mengual et al. reported on 12+2 mRNA genes (ANXA10, AHNAK2, CTSE, CRH, IGF2, KLF9, KRT20, MAGEA3, POSTN, PPPIR14D, SLCIA6, TERT and ASAM, MCM10) (Clin Cancer Res. (2010) 16(9): 2624-33). However, only Holyoake et al. from New Zealand have reported on the discovery (Clin Cancer Res. 2008 Feb. 1; 14(3):742-9) and validation of five mRNA genes (CDC2, MDK, IGFBP5, HOXA13; aka Cxbladder™), with a reported sensitivity of 82% and specificity of 85%. O'Sullivan et al. J Urol. (2012) 188(3): 741-7; Kavalieris et al. J Urol. (2017) 197(6): 1419-1426. To date none have been tested in UTUC, as like voided urinary cytology, such tests will require a significant amount of exfoliated tumor cells in the urine.

Briefly, the multiplex assay described herein, a liquid biopsy for the detection and management of bladder cancer, was derived from two complementary techniques; gene expression array analysis of shed urothelial cells within urine and glycoproteomics profiling the urinary supernatant. Using sophisticated bioinformatics, the two datasets were combined, and a cancer-associated signature comprised of 19 candidate biomarkers was identified. Then, utilizing a series of validation cohorts, the 19 candidate biomarkers were reduced to 10 biomarkers: angiogenin, ANG; apolipoprotein E, APOE; alpha-1 antitrypsin, A1AT; carbonic anhydrase 9, CA9; interleukin 8, IL8; matrix metallopeptidase 9, MMP9; matrix metallopeptidase 10, MMP10; plasminogen activator inhibitor 1, PAI1; syndecan 1, SDC1 and vascular endothelial growth factor A, VEGFA and subsequently validated in several late stage studies achieving a diagnostic sensitivities of 85-93% and specificities of 81-95%. The inventors tested the performance of the multiplex assay described herein in subjects with UTUC, noting a sensitivity of 93.5 and specificity of 75.6%. The sensitivity is on par with Xpert® BC-Detection (five target mRNAs; ABL1, CRH, IGF2, UPK1B, ANXA10) which is reported at 100%, however the reported specificity is 16.7% (D'Elia et al. Ther Adv Urol. (2022) 14).

Interestingly, Table 9 depicts the diagnostic performance of the multiplex assay described herein in high-grade/low-grade and invasive/non-invasive UTUC. Regardless of grade or invasiveness, the multiplex assay described herein maintained a sensitivity above 88%. This along with its high NPV of 93.9%% would allow it to be positioned as a rule out test, i.e., a negative multiplex assay described herein would rule-out who needs cystoscopy with ureteroscopy and renal washings or biopsy.

### CONCLUSION

The diagnosis of UTUC can be difficult requiring invasive procedures to support the diagnosis of disease presence. With advancement in diagnostic technology, the development of an accurate and robust urinary test for the detection of UTUC would benefit both patients and healthcare systems. In a multi-institutional cohort study, the multiplex assay described herein achieved highly encouraging diagnostic performance. The test deploys established multiplex testing technology enabling rapid uptake in clinical laboratories.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

1. A method for predicting the likelihood of long-term survival of a bladder cancer patient comprising
(a) obtaining a biological sample from a patient;
(b) isolating mRNA from the biological sample;
(c) determining the level of the mRNA of ANG, A1AT, APOE, CA9, IL8, MMP9, MMP10, PAI-1, SDC1 and VEFGA in the biological sample;
(d) normalizing the mRNA level against a level of at least one reference mRNA transcript in the sample to provide a normalized ANG, A1AT, APOE, CA9, IL8, MMP9, MMP10, PAI-1, SDC1 and VEFGA mRNA level;
(e) comparing the normalized ANG, A1AT, APOE, CA9, IL8, MMP9, MMP10, PAI-1, SDC1 and VEFGA mRNA level to a normalized ANG, A1AT, APOE, CA9, IL8, MMP9, MMP10, PAI-1, SDC1 and VEFGA mRNA level in reference bladder tumor samples; and
(f) predicting the likelihood of long-term survival without the recurrence of bladder cancer, wherein increased ANG, A1AT, APOE, CA9, IL8, MMP9, MMP10, PAI-1, SDC1 and VEFGA mRNA levels is indicative of a reduced likelihood of long-term survival without recurrence of bladder cancer.

2-5. (canceled)

6. The method of claim 1, wherein the mRNA level is determined by microarray analysis, RNAseq, RT-PCR, RT-qPCR, quantitative PCR (qPCR), Northern blot analysis, dot blotting, Southern blot analysis, RNA sequencing, fluorescence in situ hybridization (FISH), or a combination thereof.

7. The method of claim 1, wherein the mRNA is determined by quantitative PCR (qPCR).

8-12. (canceled)

13. The method of claim 1, wherein the biological sample is blood, serum, whole, blood, circulating tumor cells, tumor cells, plasma, urine, tissue, tumor, or a combination thereof.

14. The method of claim 1, wherein the biological sample is tissue, optionally tumor tissue.

15. The method of claim 13, wherein the tissue is a fixed, wax-embedded tissue sample.

16. The method of claim 1, wherein the level of the amplicon of the RNA transcript of ANG, A1AT, APOE, CA9, IL8, MMP9, MMP10, PAI-1, SDC1 and VEFGA is represented as a threshold cycle (Ct) value and the normalized ANG, A1AT, APOE, CA9, IL8, MMP9, MMP10, PAI-1, SDC1 and VEFGA amplicon level is represented as a normalized Ct value.

17. The method of claim 1, wherein the reference bladder cancer samples comprise at least 30 bladder cancer samples.

18. The method of claim 1, wherein the method further comprises detecting and quantifying at least one additional biomarker of a urogenital-related cancer type in the biological sample or in a different biological sample.

19. The method of claim 1, wherein the method further comprises detecting and quantifying at least one additional biomarker of a different cancer type in the biological sample or in a different biological sample.

20. The method of claim 1, wherein the method is performed at several time points or intervals as part of monitoring of the subject at least one of before, during, and after treatment of the cancer.

21. The method of claim 1, wherein the method further comprising the step of preparing a report indicating that the patient has an increased or decreased likelihood of long-term survival without bladder cancer.

22-23. (canceled)

24. A method for detecting upper tract urothelial carcinoma (UTUC) biomarker comprising
(a) obtaining a biological sample from a subject;
(b) contacting a biological sample obtained from a subject with a panel of binding agents, wherein said panel comprises binding agents that bind to, and form a complex, with proteins selected from the group consisting of ANG, A1AT, APOE, CA9, IL8, MMP9, MMP10, PAI-1, SDC1, VEFGA, and combinations thereof; and
(c) detecting the presence and quantity of the protein-binding agent complexes that form in the biological sample.

25. A method of classifying test data, the test data comprising protein expression data, the method comprising:
(a) accessing, using at least one processor, an electronically stored set of training data vectors, each training data vector representing an individual cancer patient and comprising a protein expression data for the respective cancer patient, each training data vector further comprising a classification with respect to the expression level of a biomarker selected from the group consisting of ANG, A1AT, APOE, CA9, IL8, MMP9, MMP10, PAI-1, SDC1, VEFGA, and combinations thereof;
(b) training an electronic representation of a classification system, using the electronically stored set of training data vectors;
(c) receiving, at the at least one processor, test data comprising protein expression data;
(d) evaluating, using the at least one processor, the test data using the electronic representation of the classification system; and
(e) outputting a classification of the test data concerning the likelihood of upper tract urothelial carcinoma (UTUC) based on the evaluating step.

26. The method of claim 25, wherein the classification system is AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, Naïve Bayes, neural nets, penalized logistic regression, Random Forests, ridge regression, support vector machines, or an ensemble thereof.

27. The method of claim 24, wherein the classification system is an ensemble of classification systems.

28. The method of claim 24, wherein the subject was diagnosed with UTUC.

29. The method of claim 24, wherein the sample is obtained from a subject who has at least one symptom of UTUC.

30. The method of claim 24, wherein the biological sample is blood, serum, whole, blood, circulating tumor cells, tumor cells, plasma, urine, tissue, tumor, or a combination thereof.

31. The method of claim 24, wherein the biological sample is blood, urine, plasma, or a combination thereof.

32-43. (canceled)
